(19) United States
(12) Patent Application Publication
(10) Pub. No.: US 2014/0228718 A1
(43) Pub. Date: Aug. 14, 2014

(54) CRYOTHERAPY DEVICES AND METHODS TO LIMIT ISCHEMIC INJURY SIDE EFFECTS

(71) Applicant: BIOHEAT TRANSFER, LLC, Elgin, TX (US)

(72) Inventors: Kenneth R. Diller, Elgin, TX (US); Sepideh Khoshnevis, Austin, TX (US); Robert Matthew Brothers, Austin, TX (US)

(21) Appl. No.: 14/349,990

(22) PCT Filed: Oct. 4, 2012
(86) PCT No.: PCT/US2012/058706

§ 371 Date: Apr. 4, 2014

Related U.S. Application Data
(60) Provisional application No. 61/543,170, filed on Oct. 4, 2011.
(60) Provisional application No. 61/638,880, filed on Apr. 26, 2012.

(51) Int. Cl. A61F 7/10; A61N 1/04; A61H 1/00

(52) U.S. Cl. CPC A61F 7/10; A61H 1/008; A61N 1/0456; A61F 2007/0086
USPC 601/18; 607/112; 607/104; 607/3

(57) ABSTRACT

In one embodiment, an apparatus that provides cooling and mechanical and/or electrical simulation to the treated site may be used to overcome the problems associated with cryotherapy. In one embodiment, a cryotherapy device for producing a cooling effect for treating tissue includes a temperature control device which alters the temperature of at least a portion of the tissue being treated during treatment; and a blood flow device that alters the blood flow rate through the portion of the tissue being treated during and following treatment.

### STATEMENT REGARDING FEDERALLY SPONSORED RESEARCH OR DEVELOPMENT

This invention was made with government support from the National Science Foundation, Grant number CBET-0966998. The U.S. Government has certain rights to this invention.

### BACKGROUND OF THE INVENTION

1. Field of the Invention

The invention generally relates to devices and methods of cryotherapy. More particularly, the invention relates to devices and methods of cryotherapy that minimize the injury to the tissues.

2. Description of the Relevant Art

Localized cooling is commonly used after orthopedic surgery and in sports medicine to reduce bleeding, inflammation, metabolism, muscle spasm, pain, and swelling following musculoskeletal trauma and injury. The therapeutic application of cold therapy has a long history dating from the time of Hippocrates and is widely practiced. Over the past two decades the use of cryotherapy has increased dramatically with the advent of cryotherapy devices. Generally a cryotherapy device includes an insulated container filled with ice and water and a submersible pump to propel the flow of ice water through a cooling bladder applied to a therapy site. These devices are now used prescriptively on nearly all orthopedic surgery procedures and in many sports and rehabilitation medicine settings, numbering at least several million applications annually. Nonetheless, there remains to the present time considerable controversy over the appropriate protocol for application of cryotherapy. One extreme camp advocates continuous use of cryotherapy to a treatment site with no break in cooling for days or even weeks, whereas other practitioners recommend a maximum application duration of 20 to 30 minutes followed by a cessation period of about 2 hours. Although continuous cooling appears to be tolerated by many patients, there have been a large number of reported incidences in which continuous application of a cryotherapy device led directly to extensive tissue necrosis and/or nerve injury in the treatment area, sometimes with dire medical consequences.

It is well known that a lowered tissue temperature depresses the conduction velocity in nerves, which, in combination with local ischemia, is thought to relate to incidence of nerve injury. Cell necrosis can be due to a number of complicating factors precipitated by cold-induced ischemia. It has been known for many years that reduced temperatures cause a local decrease in blood perfusion of tissues. On the one hand, temporarily reduced blood flow can be of advantage in treating soft tissue injuries. But, when a prolonged state of ischemia is maintained, the consequences of an insufficient supply of cell nutrients in conjunction with the buildup of metabolic byproducts may lead directly to tissue necrosis and neuropathies. Causation of a prolonged state of ischemia can lead to the occurrence of reperfusion injury when blood flow is reestablished to the affected tissue. In some cases, these types of injuries are the unfortunate byproduct of the application of cryotherapy. Thus, there is a need to achieve a balance between deriving the benefits of applied cryotherapy while reducing the risk of causing further injury to the tissue being treated.

### SUMMARY OF THE INVENTION

The embodiments described herein address the use of cryotherapy devices and the direct relation to why cryotherapy injuries occur and also prevent the persistent ischemia that develops during cryotherapy which can lead to necrosis and neuropathy, especially for the extended exposure periods that are often prescribed.

Localized cooling of tissue is widely used in treating soft tissue injuries for controlling pain and inflammation. In some cases cryotherapy can also be causative to tissue necrosis and neuropathy via cold-induced ischemia. There is an explicit need for a cryotherapy device that can be applied in a manner that does not starve tissues of oxygen and nutrients and does not allow an accumulation of toxic metabolic byproducts, all of which can lead to cell and nerve death. Large numbers of these types of complications have been reported in conjunction with the use of cryotherapy units.

Although nonfreezing cold injuries have been recognized for many years, primarily in the context of military medicine, the governing mechanism of cryotherapy injury has only recently been clarified and quantified. We have found that lowering tissue temperature causes a major loss of blood flow in the affected area, known as ischemia, that persists long (many hours) after the cessation of surface cooling and the rewarming of the tissue. The persistence of ischemia deprives the tissue and nerves of their supply of oxygen and nutrients, and it allows the buildup of toxic metabolic by products, both of which lead to cell damage.

The embodiments described herein inhibit and/or do not allow the development of a state of persistent ischemia in the treatment area. The embodiments may be adopted for application with existing technologies or may be implemented with a more sophisticated and accurate means of skin temperature control with the objective of preventing reduced blood flow or to stimulate blood flow. Embodiments of the present invention may further embody a thermal barrier designed for explicit regulation of the skin temperature, and in each event retains the benefits of the practice of cryotherapy.

### BRIEF DESCRIPTION OF THE DRAWINGS

FIGS. 1-3 depict temperature and perfusion data for various cryotherapy trials;

FIG. 4 depicts the extent to which an ischemic state is induced with respect to time over which cryotherapy is applied;

FIG. 5 depicts an embodiment of a cryotherapy device;

FIG. 6 depicts a device for controlling the blood flow rate through the targeted tissue by mechanical stimulation;

FIG. 7 depicts an alternate device for controlling the blood flow rate through the targeted tissue by mechanical stimulation;

FIG. 8 depicts a device for controlling the blood flow rate through the targeted tissue by electrical stimulation;

FIG. 9 depicts a cryotherapy device that includes a heat transfer substrate and a heating element coupled to the heat transfer substrate;

FIG. 10 depicts a ribbon cryotherapy device;

FIG. 11 depicts a cryotherapy device that includes a plurality of sensors used to monitor the underlying tissue during treatment;

FIG. 12 depicts laser doppler blood flow histories during cryotherapy of the knee;

FIG. 13 depicts data showing increase in blood perfusion during transdermal electrical nerve stimulation;

FIG. 14 depicts a cryotherapy device that provides mechanical stimulation for controlling the blood flow rate through the targeted tissue by a mechanical impulse to glabrous skin;

FIG. 15 shows the effect of flavanol supplementation during cryotherapy;

FIGS. 16A-B depict an embodiment of a cryotherapy device for a knee;

FIGS. 17A-B depict data for a cryotherapy trial with a Don Joy Iceman 1100 system applied to the shin;

FIGS. 18A-B depict data for a cryotherapy trial with a Breg Polar Care 500 Lite system applied to the knee;

FIGS. 19A-B depict data for a cryotherapy trial with a DeRoyal T600 applied to the shoulder;

FIGS. 20A-B depict data for a cryotherapy trial with an Arctic Ice System applied to the calf and shin area of the lower leg;

FIGS. 21A-B depict data for a cryotherapy trial with an Arctic Ice System applied to the calf and shin area of the lower leg;

FIGS. 22A-B depict data for a cryotherapy trial with an Arctic Ice System applied to the calf area of the lower leg;

FIGS. 23A-B depict data for a cryotherapy trial with a Game Ready cryotherapy unit applied to the knee;

FIGS. 24A-B depict data for a cryotherapy trial with an Aircast Cryo/Cuff cryotherapy unit applied to the knee;

FIGS. 25A-B depict data for a cryotherapy trial with a Breg Polar Care 300 cryotherapy unit applied to the knee;

FIGS. 26A-B depict data for a cryotherapy trial with a DonJoy Iceman 1100 unit applied to the ankle;

FIGS. 27A-B depict a control experiment with full instrumentation and the Breg Polar Care 500 Lite cooling bladder applied to the knee;

FIGS. 28A-B depict data for an alternate cryotherapy trial with a Breg Polar Care 500 cryotherapy unit applied to the knee;

FIG. 29 depicts infrared thermograph of the temperature distribution on the surface of a Breg Polar Care 300 knee pad during perfusion with ice water;

FIG. 30 depicts a plot of minimum skin temperature vs. active cooling time;

FIG. 31 depicts cutaneous blood perfusion as a function of the active cooling time;

FIGS. 32A-B depict an alternate cryotherapy trial with a Breg Polar Care 500 cryotherapy unit applied to the knee;

FIGS. 33A-B depict data for a cryotherapy trial with a DonJoy Iceman cryotherapy unit applied to the knee;

FIGS. 34A-B depict data for an alternate cryotherapy trial with a DonJoy Iceman cryotherapy unit applied to the knee;

FIG. 35 depicts data for an alternate cryotherapy trial with a DonJoy Iceman cryotherapy unit applied to the knee; and

FIGS. 36 and 37 depict data for a cryotherapy trial with a DonJoy Iceman cryotherapy unit applied to the ankle.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

The term “cryotherapy” means local therapeutic cooling of tissue.

The term “temperature control device” means a system or apparatus with means for regulating the temperature applied to a body part of a mammal for a treatment or for obtaining a therapeutic outcome.

The term “blood flow stimulation” as used herein, refers to any device or method that causes the blood flow to increase in an area of tissue on demand Blood flow stimulation may cause blood flow to increase from an ischemic state to a normal state and to return to an ischemic state when the stimulation is withdrawn. Blood flow stimulation may be achieved by methods that include, but are not limited to: manual massage; device massage; device vibration; transient flexing of muscles; physical movement of a body part; ambulation; application of Transcutaneous Electrical Nerve Stimulation (TENS); application of Electrical Muscle Stimulation (EMS); altering the elevation of a body part; standing walking, jumping on an elastic surface; application impulse blood pump stimulation; application of sequential pneumatic compression blood pump distally, locally, or proximally; application of roller stimulation; application of active and/or passive flexion and extension on proximal and/or local and/or distal joints; stimulation distal, local and/or proximal to the cryotherapy site; local heating of tissue at the treatment site; any other method know to potentially increase local blood flow.

The term “heat transfer limiting thermal barrier”—a material positioned between a temperature control device and the portion of the tissue to cause a temperature drop between these two elements to thereby limit the rate of heat loss from the portion of tissue. The heat transfer limiting thermal barrier bay be a pneumatic compression sleeve that may be attached to a sequential compression device controller to be activated periodically to cause a temporary increase in blood flow in tissues in the treatment area.

The term “treatment temperature” means the temperature applied to a tissue or treatment area to produce a therapeutic outcome.

In a series of experiments, standard cryotherapy devices in their recommended configuration were studied on human subjects and to measure changes in skin blood flow at the application site and distally as a function of the temperature time history on the skin surface at the treatment location. Experiments were conducted on two healthy adult subjects: one male, age 68, height 1.73 m, weight 87 kg; one female, age 42, height 1.68 m, weight 53 kg. Commercially available cryotherapy devices (Breg Polar Care 500 Lite and Artic Ice System) with dedicated water perfusion bladders were used to generate cooling conditions consistent with standard cryotherapy protocols. Each device has US FDA approval, and they were used in accordance with the manufacturer's instructions. We also have tested many other cryotherapy devices, obtaining data very similar to that reported in this patent. All tests were conducted with general purpose bladders with a split shape that allowed easy conformation for placement onto a joint. These bladders are often positioned on the knee for postsurgical therapy, and the data reported herein were recorded for cooling of the knee and of the straight length of the lower leg between the knee and the ankle (calf and shin area).

Experiments consisted of placing laser Doppler flow probes, thin ribbon thermocouples, and a foil heat flux gauge directly onto the skin in the area of active cooling around the knee and, in some cases, at a distal location on the dorsal surface of the foot. The area on the skin to which the cooling bladder was applied was covered with a thermal insulation barrier according to the instructions for using the cryotherapy device. The barrier also covered all instrumentation placed directly onto the skin. Thermocouples were also placed on the surface of the cooling bladder at a location to match one of the skin temperature measurements, and in the ice water bath. In some instances the inlet and outlet water lines were cut adjacent to the bladder, and tee connectors inserted with a thermocouple placed in the side branch to monitor the water temperature immediately as it entered and left the bladder. Approximately one hour was required to place all of the instrumentation onto the subject at the beginning of an experimental session. The cooling bladder was secured in place over the instrumentation and thermal insulation barrier, and the trial was begun. Each protocol consisted of an initial period of baseline data with no flow through the bladder, followed by active cooling with flowing water, and then passive rewarming with no water flow. The cooling bladder was left in place on the subject throughout the entire test protocol.

The subject was either seated at a 75° angle or in a supine position for the duration of each experiment. All experiments were performed at a constant ambient temperature and humidity of approximately 23° C. and 60%. A light blanket was placed over the subject to eliminate any vasoactive response driven by whole body thermoregulation inputs, especially in response to a general cooling of the overall skin surface.

Skin blood flow was monitored with two laser Doppler flow systems: a Moor Instruments VMS-LDF2 two channel perfusion and temperature system and a Moor Instruments VMS-LDF1-HP single channel high power perfusion probe (Moor Instruments, Oxford, UK). The LDF2 device supports two fiber optic probes that incorporate both laser Doppler and optical temperature measurement. The flow field sampling depth is relatively superficial, centered at about 0.5 mm into the skin, extending from near the surface to about 2 mm. The LDF1-HP device incorporates only a single perfusion probe, but with a higher power laser and a wider sensor separation resulting in a deeper sampling depth centered at about 2 mm, extending from near the surface to about 4 mm. The probe heads are affixed to the skin via a flexible probe holder and a double-sided adhesive disc. The probe heads incorporate the fiber optic and sensor surface in a right angle configuration that stands about 1 cm proud of the skin surface. Consequently, when these probes are positioned on a surface that underlies the cooling bladder, the cold source is held away from the skin for an area about 2 cm in diameter resulting in temperatures somewhat higher than in regions where the bladder directly contacts the thermal barrier material.

Temperatures were monitored with ribbon type T (copper/constantan) thermocouples (Omega Engineering, Stamford, Conn.). These sensors have a flat thermal element that is 25 μm thick that is embedded in a thin polyimide sheath that is taped to the skin surface peripheral to the sensing element. The typical time constant for these devices is on the order of 0.1 s, which is much shorter that necessary to follow the temperature transients for cryotherapy experiments. The primary advantages of these sensors is the low mass and thin cross section that causes minimal perturbation to the temperature field, and their mechanical flexibility that allows them to conform to the surface contours of the skin. Multiple thermocouples, typically at least six, were affixed to the skin at locations beneath the cooling bladder as well as at proximal and/or distal sites. In addition, a heat flux gauge (Omega Engineering) was mounted to the skin beneath the cooling bladder to provide a continuous measure of the heat flow at the skin surface. The heat flux gauge was also fabricated in a thin polyimide sheath, and it included an embedded type K (chromel/alumel) thermocouple.

All electrical sensor outputs (fiber optic blood perfusion and temperature, thermocouples, heat flux gauge) were read into a data acquisition interface (NI 9201, 9211, and 9213; National Instruments, Austin, Tex.) and recorded using NI LabVIEW Signal Express software. Data was sampled at 12 Hz, and the thermocouple and heatflux inputs were down sampled to 3 Hz. Data files subsequently were transferred to a host computer where they subjected to processing and plotting using MATLAB (MathWorks, Natick, Mass.). Periodic manual measurements of the subject blood pressure were performed using a sphygmomanometer and stethoscope. These values were applied to normalize the blood perfusion measurements to calculate values for cutaneous vascular conductance (CVC) as CVC=laser Doppler flux*100/mean arterial pressure.

Prior to the start of a measurement protocol, room temperature water was pumped through the cooling bladder, and all air was bled from the flow lines via visual inspection. The room temperature water remained captured within the bladder until the cooling process was commenced by the pumping of ice water. After all of the instrumentation was in place and its function verified, the cooling bladder was applied. Each protocol was initiated with a period of data acquisition before the cryotherapy device water pump was turned on. Typical durations of this period ranged from 10 to 45 minutes. The purpose of this period was to obtain baseline data for perfusions and temperatures to which subsequent changes elicited by the cryotherapy procedure could be normalized. Although the values of temperature and especially blood perfusion were never perfectly static, variations were small in comparison with the magnitude of variations caused by the cooling procedures.

The cooling process was initiated at the end of baseline data collection by turning on the pump to produce a steady flow of ice water through the bladder. The duration of the cooling period was a primary control parameter evaluated in this study. Ice water flow through the bladder was maintained continuously for a wide range of periods, including 1, 2, 3, 5, 10, 20, 30, 60, and 90 minutes.

The cooling process was terminated by turning off the ice water pump, following which both the bladder and the underlying tissues began to warm via a combination of parasitic heat gain from the environment and active warming from within the tissues by metabolism and convection from blood. Both of the latter effects were depressed at the lower local tissue temperatures. The period of time over which the rewarming process was monitored also was varied over a wide range of values from 30 to 240 minutes. One of the primary determinates of the period of data collection during rewarming was the extent to which the blood perfusion may have returned toward the baseline value. In some trials cooling was reinitiated following a defined period of rewarming. The cooling and rewarming cycle could be repeated multiple times.

Control data was obtained by pumping thermally neutral water at skin temperature through the bladder to determine whether the mechanical pressure owing to water flow caused an effect on the blood perfusion. All parameters of the cryotherapy protocol with the exception of the water temperature were the same for the control trials.

A typical data set from an experimental trial consists of a set of two plots of temperature and blood perfusion measurements at specific sites under the cooling bladder and at proximal and/or distal sites that were not cooled directly. For trials lasting as long as six hours and involving data acquired from 10 or more sensors at 3 Hz or greater, the amount of information generated is large. As would be anticipated, data is not reproduced exactly among the various trials, but certain very repeatable behaviors have been identified. The results are presented in a format to emphasize the primary and most important phenomena that have been measured and characterized.

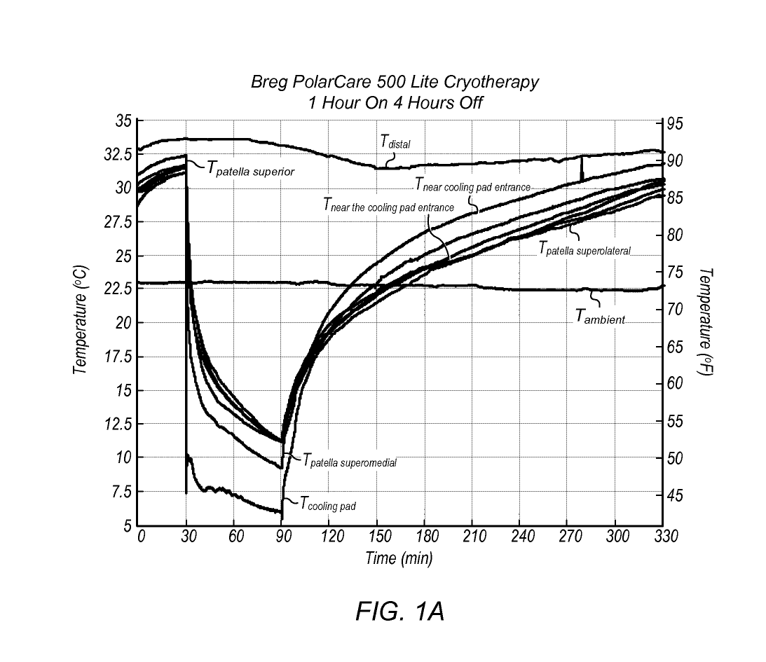

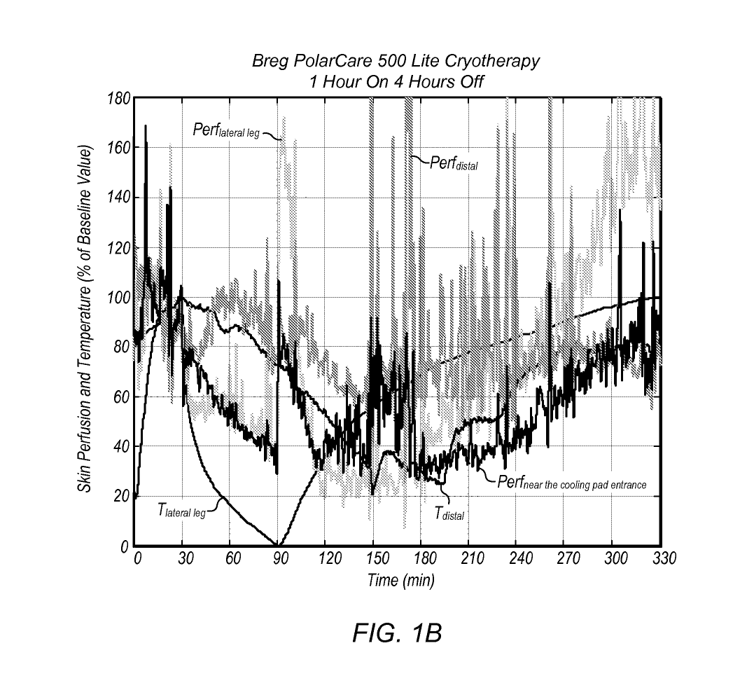

FIGS. 1-3 present temperature and perfusion data respectively for: a relatively long single period of cooling followed by a long rewarming period; repeated short cooling periods followed by relatively short rewarming periods; and skin temperature water flowing through the cooling bladder.

FIGS. 1A and 1B depict data for a cryotherapy trial with a Breg Polar Care 500 Lite system consisting of 30 minutes of baseline data, followed by active cooling for 60 minutes and passive rewarming for 240 minutes. In FIG. 1A, temperature histories were measured at four locations under the cooling bladder, on the surface of the bladder, and on the dorsal surface of the foot. In FIG. 1B, superficial (green) and deep (red) perfusion histories under the cooling bladder on the knee and superficial perfusion (magenta) on the dorsal surface of the foot are shown. Also, temperatures measured optically at the two superficial perfusion sites are plotted on a normalized scale.

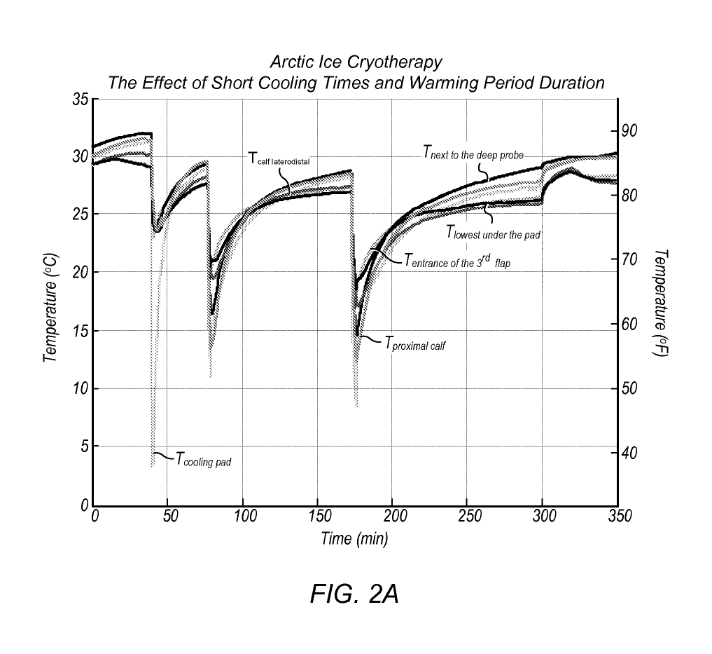

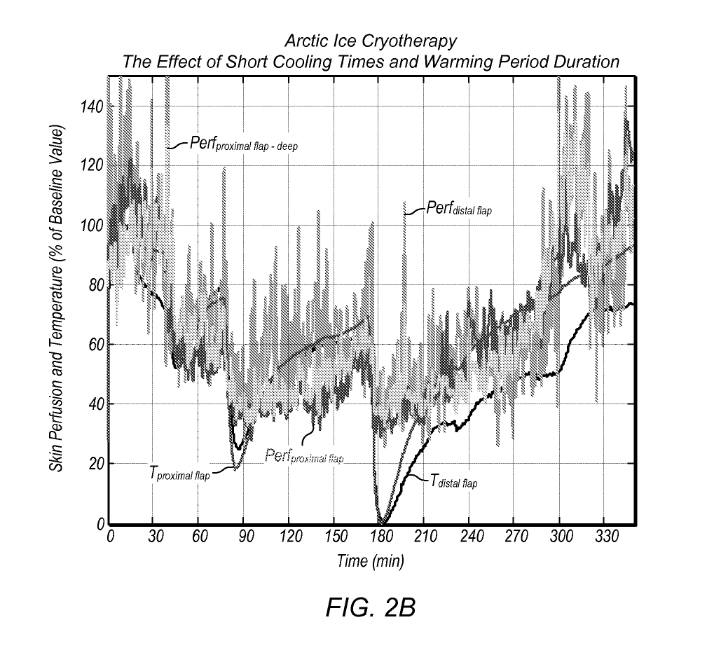

FIGS. 2A and 2B depict data for a cryotherapy trial with an Arctic Ice System applied to the calf area of the lower leg, consisting of 39 minutes of baseline data, followed by active cooling for 1 minute and passive rewarming for 37 minutes, then 2 minutes of active cooling and 96 minutes of passive rewarming, then 3 minutes of active cooling and 122 minutes of passive rewarming FIG. 2A shows temperature histories measured at five locations under the cooling bladder and on the surface of the bladder. In FIG. 2B, superficial (green and black) and deep (red) perfusion histories under the cooling bladder. Also, temperatures measured optically at the two superficial perfusion sites are plotted on a normalized scale.

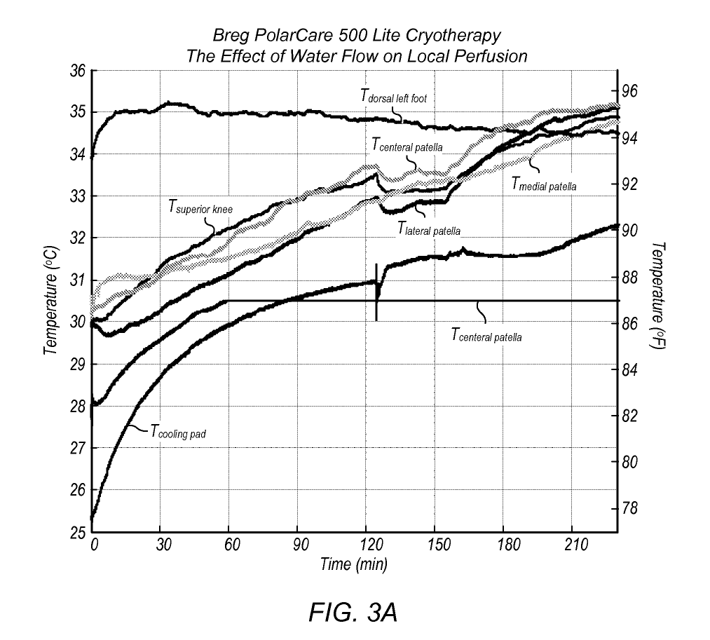

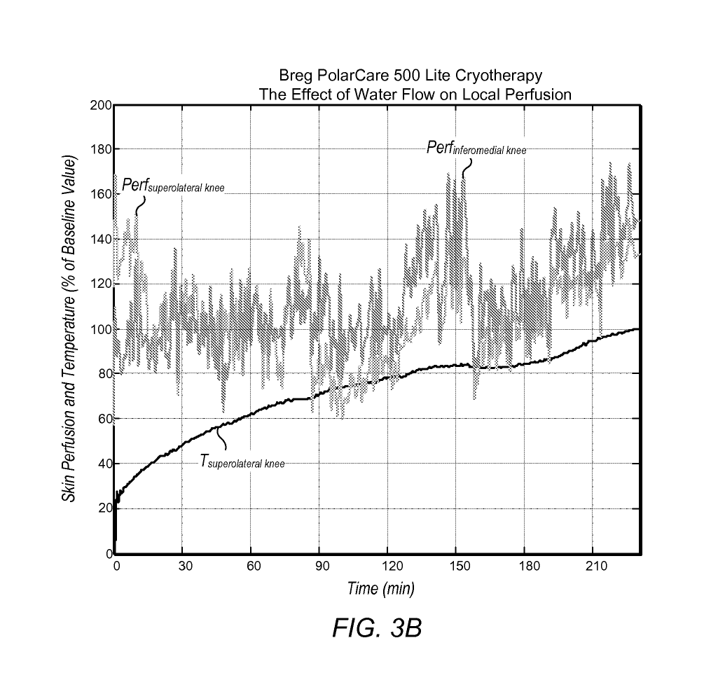

FIGS. 3A and 3B depict a control experiment with full instrumentation and the Breg Polar Care 500 Lite cooling bladder applied to the knee. Only thermally neutral water was pumped through the bladder. FIG. 3A depicts temperature histories measured at five sites under the cooling bladder, on the surface of the bladder, and on the dorsal surface of the foot. Skin temperature water was perfused through the bladder from 125 to 155 minutes. There was no flow through the bladder at other times. The bladder and water contained therein were initially at room temperature (23° C.) when it was applied to the knee. FIG. 3B depicts skin blood perfusion measured at two locations under the cooling bladder. The probe locations and distinction between deep (red) and superficial (green) measurements are denoted. Also, temperatures measured optically at the two superficial perfusion sites are plotted on a normalized scale.

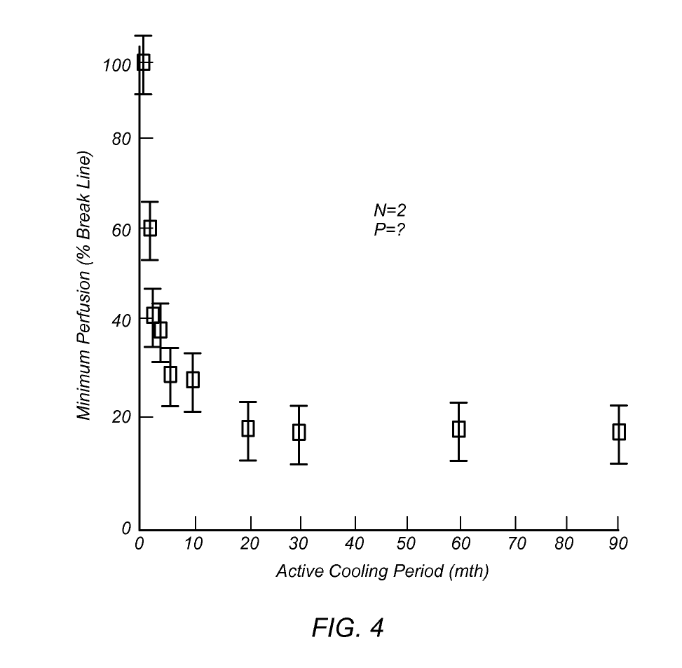

FIG. 4 depicts the extent to which an ischemic state is induced is dependent upon the time over which cryotherapy is applied. The vertical axis depicts the maximum reduction in local blood perfusion achieved during the total cooling and rewarming cycle.

The results demonstrate the even short episodes of cryotherapy can produced a prolonged duration of ischemia in the local area of treatment as well as in more distal tissues. The state of ischemia endures long after local temperatures have rewarmed toward the normal range, indicating the ischemia is not directly coupled to a cold state. It is likely that a long acting humoral vasoconstrictive agent is released locally that continues to be active long after tissue temperatures have rewarmed toward their baseline values. Evidence for the action of such vasoconstrictive agents has been confirmed in the occurrence of nonfreezing cold injury. The suppression of blood perfusion at sites distal to the site of applied cryotherapy may be a consequence of the vasoconstrictive agent eventually washing downstream in the residual blood flow, causing distal vasoconstriction in tissues that were not affected directly by the cryotherapy.

In one embodiment, an apparatus that provides cooling and mechanical and/or electrical simulation to the treated site may be used to overcome the problems associated with cryotherapy.

In one embodiment, a cryotherapy device for producing a cooling effect for treating tissue includes a temperature control device which alters the temperature of at least a portion of the tissue being treated during treatment; and a blood flow device that alters the blood flow rate through the portion of the tissue being treated during and following treatment.

In one embodiment the temperature control device includes a heat transfer substrate which receives a cooling fluid during treatment of the portion of the tissue and transfers heat between the cooling fluid and the tissue. In an alternate embodiment, the temperature control device is composed of a thermoelectric material, and wherein the temperature of the thermoelectric material is altered by applying a voltage to the thermoelectric material. The cryotherapy device may also include a voltage source capable of supplying alternating applied voltage to thermoelectric material to create alternate heating and cooling of the portion of the tissue.

In one embodiment, the blood flow device provides mechanical stimulation to the tissue to increase the flow of blood through the portion of the tissue. In an embodiment, the mechanical stimulation is accomplished by an impulse to glabrous skin at the end of an appendage being treated. In an alternate embodiment, the blood flow device provides electrical stimulation to the tissue to increase or decrease the flow of blood through the portion of the tissue.

In an alternate embodiment, the temperature control device includes multiple segments that include a thermoelectric material. Each of the segments may be controlled individually, the segments being mounted on a flexible substrate that is capable of conforming to the surface geometry of the portion of the tissue. The multiple thermoelectric segments may be arranged and controlled so as to produce a temperature grid of cooler and warmer regions on the portion of the tissue that can be modulated in both position and time so as to produce a desired therapeutic cooling effect with reduced risk of causing induced injury to the treatment and adjacent areas.

The cryotherapy device may include a variety of sensors to monitor the conditions of the tissue being treated. For example, the cryotherapy device may include: one or more temperature sensors couplable to the tissue to measure the surface temperature of the tissue; one or more blood flow rate sensors couplable to the portion of the tissue to measure blood perfusion in the tissue; and one or more oxygenation sensors couplable to the portion of the tissue to measure the level of oxygenation in the tissue. The cryotherapy device may also include a controller coupled to one or more of the temperature sensors, one or more of the blood flow rate sensors and one or more of the oxygenation sensors. The controller may use the data collected from one or more of the sensors to modulate the operation of the temperature control device and the blood flow device to achieve a desired therapeutic outcome.

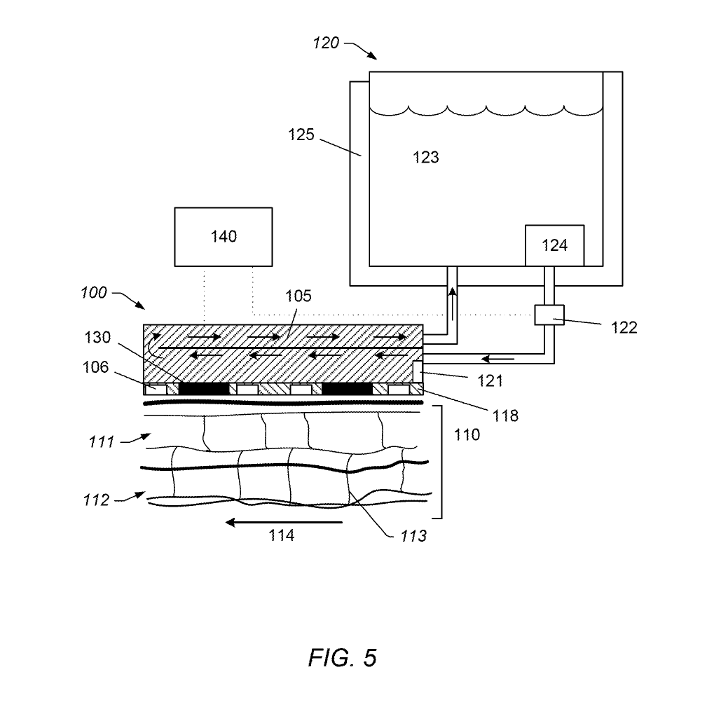

An embodiment of a cryotherapy device is depicted in FIG. 5. FIG. 5 depicts cryotherapy device 100 that provides cooling to a portion of the body and includes a blood flow device to alter the flow rate of blood through the body portion. Cryotherapy device 100 is coupled to tissue 110. Tissue 110 is composed of the epidermis and dermis (layer 111) and the subcutis 112. The tissue area includes vasculature 113 extending through the tissue 110. The direction of blood flow through the tissue is generally depicted by the arrow 114.

Cryotherapy device 100 includes a heat transfer substrate 105 that receives a cooling fluid (depicted as a flow of arrows through the substrate 105) during treatment of a portion of the tissue. An insulating layer 118 is coupled to the heat transfer substrate 105 to protect the underlying tissue from the cold substrate. Temperature sensors 106 may be disposed in insulating layer 118 to measure the skin temperature. A temperature sensor 121 is included to monitor the temperature of the incoming fluid.

Cryotherapy device 100 may also include one or more blood flow devices 130 that alter the flow of blood through the portion of the tissue being treated during and following treatment. Blood flow devices 130 may be mechanical and/or electrical stimulation devices.

Substrate 105 receives a cooling fluid from cooling fluid supply source 120. Cooling fluid supply source 120 may a refrigerated cooling device capable of providing cooling water. Cooling fluid supply source 120 includes an insulation layer 125 around the cooling supply source. Cooling fluid 123 is sent to cryotherapy device 100 using pump 124.

A controller 140 is coupled to blood flow devices 130 and control valve 122. Controller 140 may control the operation of the blood flow devices to increase the blood flow through the portion of the tissue, based on data collected by monitoring the tissue sample. Controller 140 may also control the fluid flowing through substrate 105 by operating control valve 122. By controlling both the temperature of the tissue sample and the blood flow through the tissue sample, cryotherapy may be achieved while reducing the incidence of edema and prolonged ischemia in the portion of tissue being treated.

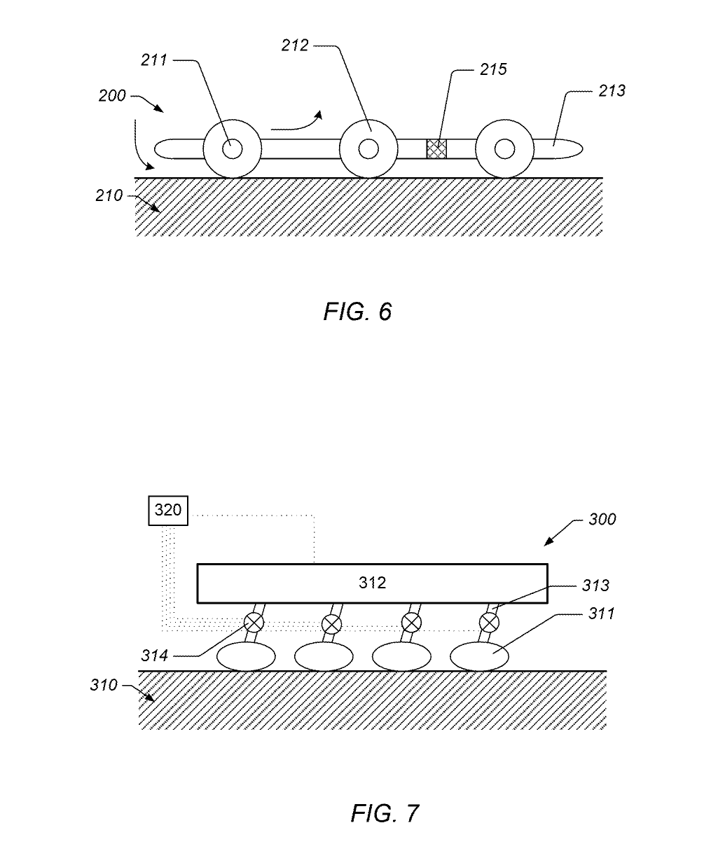

FIG. 6 depicts a device 200 for controlling the blood flow rate through the targeted tissue by mechanical stimulation of the targeted tissue and/or proximal tissue and/or distal tissue. Device 200 includes a frame 213 that holds one or more rollers 212, coupled to the frame through axels 211. Device 200 also includes pressure sensor 215 which determines the pressure being applied to the tissue 210 being treated. Device 200 may move across the surface of the targeted tissue to produce a peristaltic pumping action on the blood in the tissue.

FIG. 7 depicts an alternate device 300 for controlling the blood flow rate through the targeted tissue by mechanical stimulation of the targeted tissue and/or proximal tissue and/or distal tissue. Device 300 includes pneumatic bladders 311 which provide varying pressure to tissue 310 being treated. Bladders 311 are coupled to a pressurized air source 312 via conduits 313. Pneumatic bladders 311 provide mechanical stimulation to the tissue due to inflation/deflation cycles which are controlled by controller 320 via valves 311. During use, controller 320 provides control signals to active valves 311 to send air into bladders 311 or to release air from the bladders to create mechanical stimulation of the tissue. The control signals may be coordinated to cause an overall movement of blood within the target tissue.

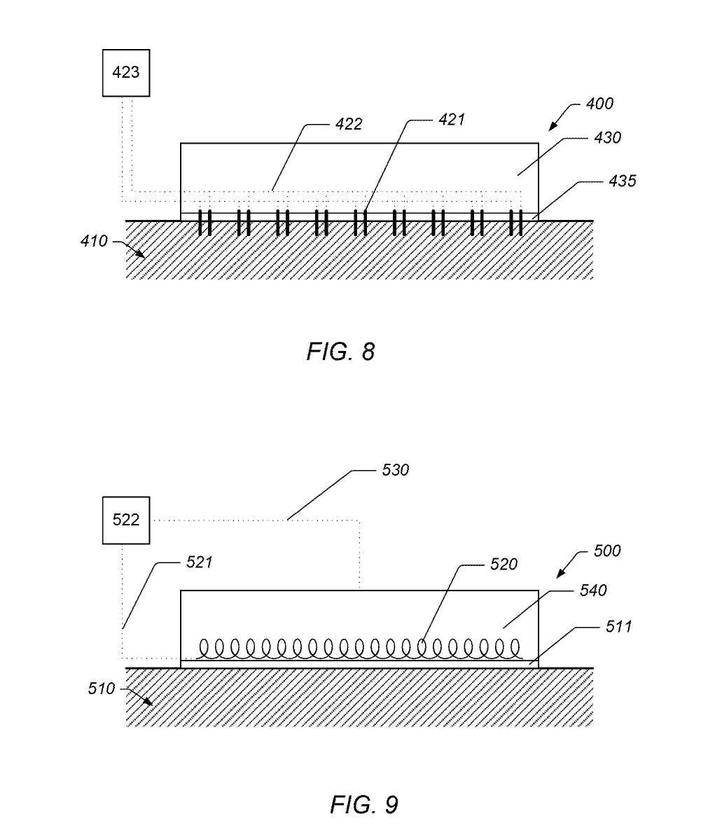

FIG. 8 depicts an alternate device 400 for controlling the blood flow rate through the targeted tissue by electrical stimulation of the targeted tissue and/or proximal tissue and/or distal tissue. Device 400 includes one or more pairs of electrodes 421 coupled to a heat transfer substrate 430 and also coupled to the surface of the skin the targeted tissue. In some embodiments, electrodes 421 are disposed in thermal insulation layer 435. Electrodes 421 may be used to provide electrical stimulation to the underlying tissue 410 to alter the blood flow rate. Controller 420 controls the electrical stimulation provided from electrodes resulting in control of the state of vasoconstriction and vasodilation of the tissue vasculature. Electrodes 421 are coupled to controller 423 via wires 422.

FIG. 9 depicts a cryotherapy device 500 that includes a heat transfer substrate 540 and a heating element 520 coupled to the heat transfer substrate. In an embodiment, heat transfer substrate 540 provides cooling to the tissue 510 using a cooling fluid or other means. Heating element 520 may be used to provide heat to the underlying tissue 510. Controller 522 is coupled to the heat transfer substrate 540 via wire 530, and coupled to heater 520 via wire 521. Controller 522 includes a timer for programmed activation of heating and/or cooling of the tissue.

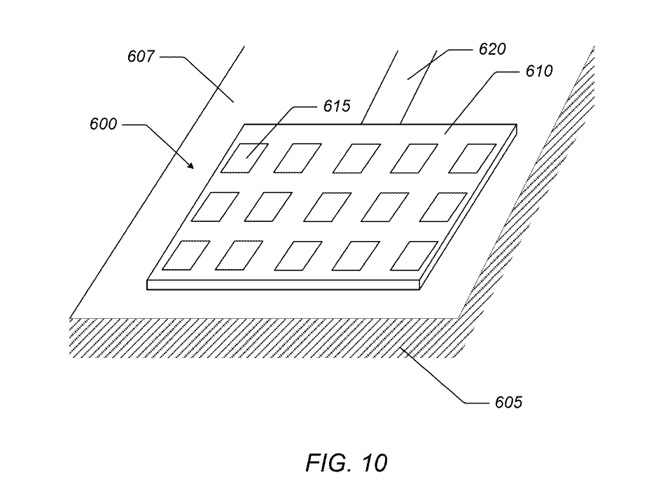

FIG. 10 depicts an alternate embodiment of a ribbon cryotherapy device 600. Cryotherapy device 600 includes a flexible, heat conductive substrate 610 to which a plurality of thermoelectric segments 615 are mounted. In an embodiment, thermoelectric segments 615 may be mounted in an array, as shown in FIG. 10. Thermoelectric segments 615 are formed from a thermoelectric material which allows the temperature applied to the tissue to be controlled by an applied voltage. The voltage applied to the thermoelectric segments 615 can be modulated over time to alternate cooling and heating. In some embodiments, each of thermoelectric segments 615 can be controlled individually to provide a pattern of heating and/or cooling to the underlying tissue. A controller is coupled to each of the thermoelectric segments through coupling 620.

The multiple thermoelectric segments 615 are arranged and controlled so as to produce a temperature grid of cooler and warmer regions on the treatment area that can be modulated in both position and time so as to produce a desired therapeutic cooling effect with reduced risk of causing induced injury to the treatment and adjacent areas. The period of time during which the temperature of a region is raised is small in comparison to the time constant for heat diffusion into the tissue so as to limit the overall loss of therapeutic cooling benefit but is sufficiently long to produce a heating of the superficial layer of skin so as to elicit a temporary increase in blood perfusion through that region of skin. The controller operates each of the thermoelectric segments to regulate the temperature magnitude, frequency, and duration of heat and/or cooling.

In some embodiments, each thermoelectric segment may be controlled individually to create a predetermined pattern of cooling and/or heating of the underlying tissue. In this manner cooling, for example, can be restricted to the portion of the tissue that will benefit from cooling, while minimizing the amount of cooling to the tissue surrounding the affected area. To improve circulation around the affected tissue, some thermoelectric segments surrounding the cooling thermoelectric segments may be activated. Thus, the combination of targeted cooling and targeted heating may provide the benefits of increased blood flow to the affected tissue, while minimizing the area being treated. The cooling/heating pattern of the thermoelectric sensors may be controlled through a controller. The controller may have predetermined patterns, designed for specific areas of the body. The controller may also be customizable to allow customized patterns of cooling and heating.

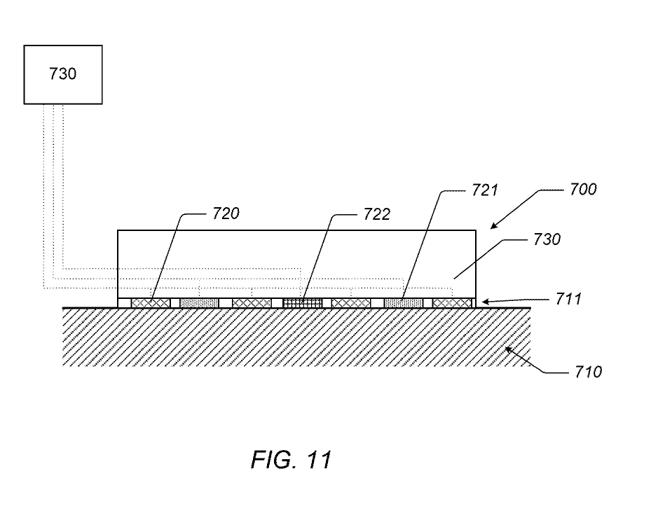

FIG. 11 depicts a cryotherapy device 700 that includes a plurality of sensors used to monitor the underlying tissue 710 during treatment. Cryotherapy device 700 includes a heat transfer substrate 730 with an insulating layer 711. One or more sensors (720, 721, and 722) are coupled to the controller 730 for monitoring the tissue. Examples of sensors that may be used include a temperature sensor 720 used to monitor tissue surface temperature. A blood perfusion sensor 721 may also be may also be present to measure the blood flow rate through the tissue. Additionally, a tissue oxygenation sensor 722 may also be used to monitor the oxygen level of the tissue. Controller 730 may control the operation of cryotherapy device 700 based on information collected from the sensors.

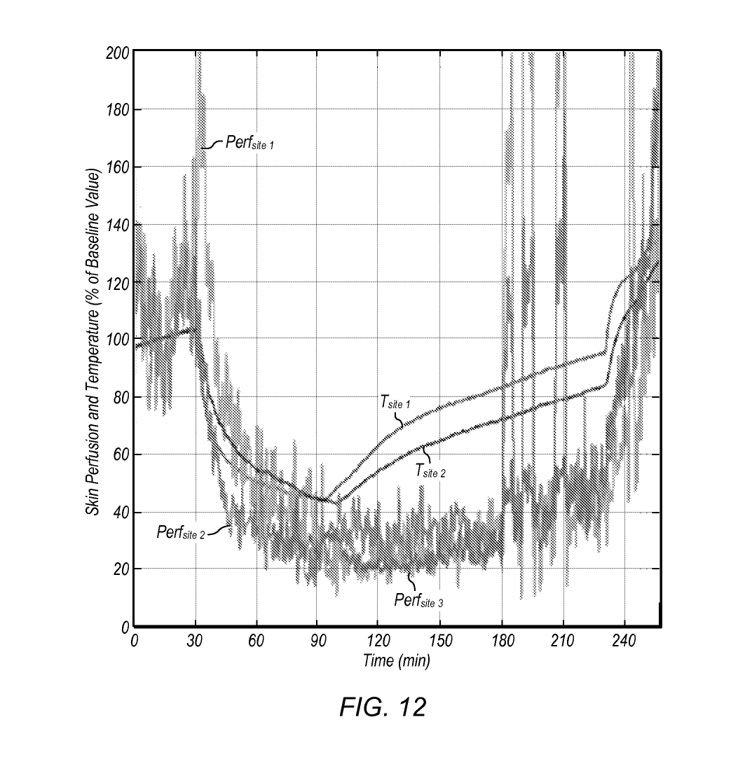

FIG. 12 depicts laser doppler blood flow histories during cryotherapy of the knee. An exemplar plot of blood flow data for a cooling trial is shown. Data are from two shallow probes (0.5 mm, green, magenta) and one deep probe (2 mm, red). Temperature plots are not to scale. Blood flow stimulation started at 180 min. The protocol consisted of 30 minutes baseline with the cooling bladder in place by no water flow, 60 minutes of active cooling, 90 minutes of passive warming, three episodes of mechanical stimulation of the foot, and 30 minutes of active heating with warm water flowing through the bladder. The data are instructive to several important phenomena. When cooling is initiated there is a brief spike in the perfusion values in response water flowing through the bladder. Thereafter follows a rapid drop in perfusion to about 25% baseline that continues unhindered after the completion of cooling. At 180 min gentle manual manipulation of the foot was started for five minutes and then discontinued for three identical episodes. Blood flow immediately increased when manipulation was started and decreased when it was stopped. The effect was largest for the probe closest to the foot and diminishes with distance. Local warming caused an immediate increase in all three perfusion measurements. Thus, this data demonstrates the profound and prolonged cold induced ischemia, plus independent mechanisms to override it.

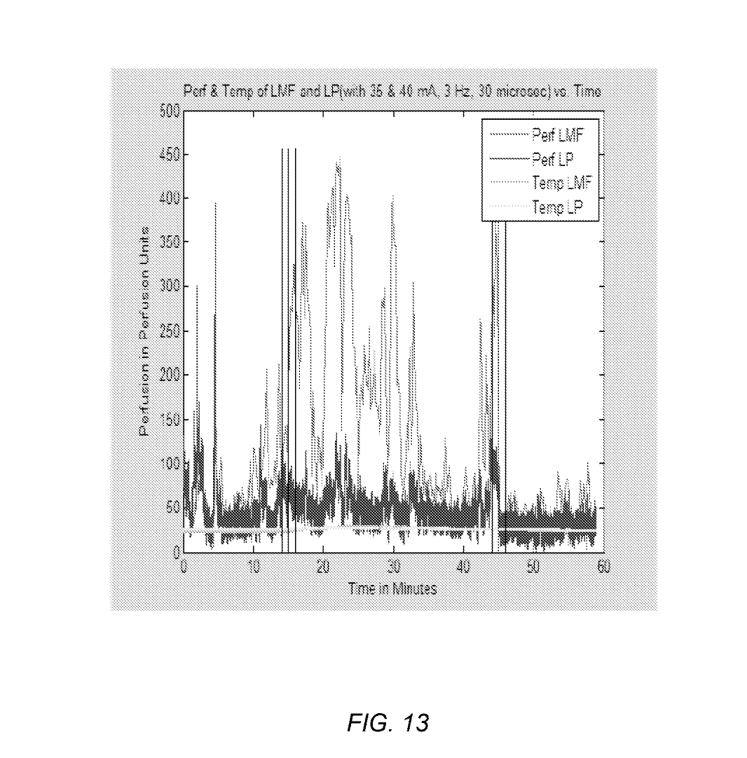

FIG. 13 depicts data showing increase in blood perfusion in the palm (blue) and middle finger pad (red) during transdermal electrical nerve stimulation (TENS) between 15 and 45 minutes during an experiment on a human subject. In this trial the electrical stimulation was applied with a current in the range of 35-40 milliamps, a pulse frequency of 3 Hz, and a pulse duration of 30 microseconds. Blood flow increased by a factor of between two and ten during stimulation in comparison to baseline.

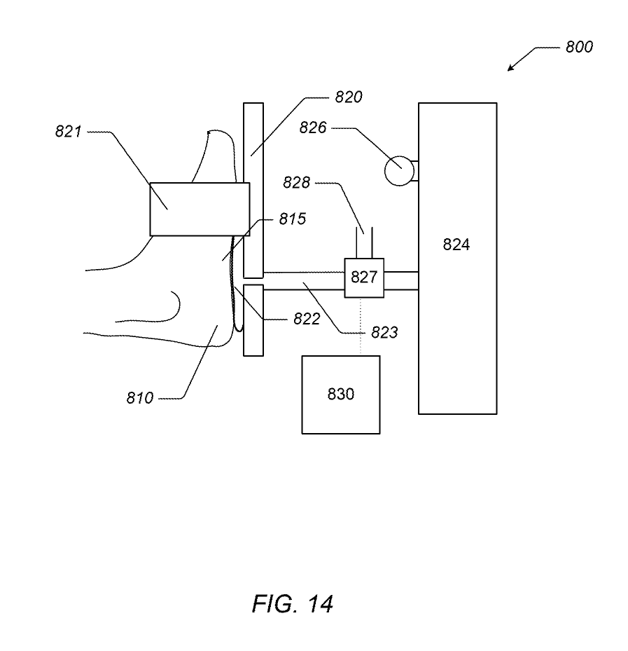

FIG. 14 depicts a cryotherapy device 800 that provides mechanical stimulation for controlling the blood flow rate through the targeted tissue by a mechanical impulse to glabrous skin at the end of an appendage being treated, such as the sole of the foot and the palm of the hand. In FIG. 14 a glabrous section of tissue 810 is coupled to cryotherapy device 800 using strap 821. Appendage 810 is coupled to rigid backing surface 820 with pneumatic bladder 822 disposed between the glabrous skin 815 and the backing surface. Bladder 822 is coupled to air source 824 via conduit 823 and valve 827. Air source 824 includes a pressure regulator 826 which controls the air pressure inside the air source. Controller 830 is coupled to valve 827 to control air flow through the device. In use, bladder 822 is filled with air, or air is removed from bladder 822 to create pulsed application of mechanical stimuli to appendage 810. In some embodiments, valve 827 is a three way valve that allows controlled release of air from bladder 822 through conduit 828.

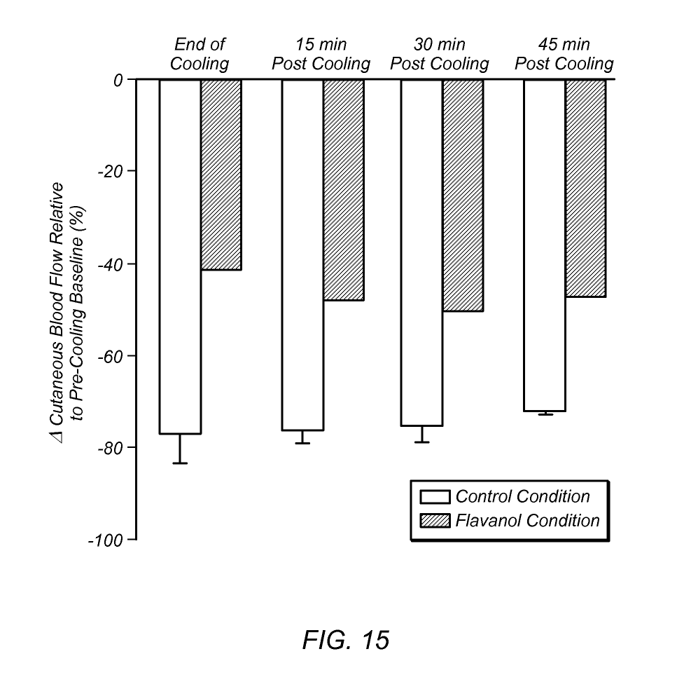

In an embodiment, a method for reducing the risk of cold-induced ischemic injury during cryotherapy is accomplished by administering a composition that includes flavanols to a subject in need of cryotherapy, wherein the administered flavanols increase the bioavailability of nitric oxide, thereby attenuating the extent of vasoconstriction caused by local cooling of tissue. Data in FIG. 15 shows the effect of flavanol supplementation during cryotherapy on causing a 40% reduction in the extent of vasoconstriction during the period of active cooling and the subsequent period of passive rewarming during which profound vasoconstriction persists for hours. The flavanol beverage was consumed two hours prior to the end of active cooling, matching the period for which it is thought to have its maximum effect in decreasing the production of free radicals that diminish nitric oxide (NO) availability. NO is a powerful vasodilator that is suppressed during cryotherapy, resulting in a deep state of ischemia.

Flavanols are a subfamily of flavonoids found in vegetables, fruits, wine, tea, and cocoa which that act as classic antioxidants to scavenge free-radicals. High levels of free radicals, especially superoxide, can reduce the bioavailability of NO and thus any NO mediated actions. Acute flavanol supplementation decreases production of free radicals which result in increased NO mediated vasodilation. The data in FIG. 15 depicts cryotherapy testing on a human subject that shows that the cold-induced cutaneous vasoconstriction was attenuated by approximately 40% when the cold was preceded consumption of a cocoa containing beverage that had high flavanol content.

Experiments. Commercially available cryotherapy devices (Breg Polar Care 300, 500, 500 Lite; DeRoyal T600, T505; Game Ready; DonJoy Ice Man 1100; Artic Ice System, Aircast Cryo/Cuff) with dedicated liquid perfusion bladders were used to generate cooling conditions consistent with typical cryotherapy protocols. All tested devices either have US FDA approval or are exempt and were used in accordance with the manufacturers' instructions. We also have tested other cryotherapy devices, obtaining data consistent with that reported in this paper. The data reported herein were recorded for cooling of the knee, the shoulder, and the straight length of the lower leg between the knee and the ankle (calf and shin area).

The subjects were either seated with their back at a 45° angle from horizontal and the legs extended on the same plane as the hips or reclining in a supine position for the duration of each experiment. Physical movements of the limb being tested were kept to a minimum. All experiments were performed at a constant ambient temperature and relative humidity of approximately 23° C. and 60%. A light blanket was placed over the subject, at his/her request, to eliminate any vasoactive response driven by whole body thermoregulation inputs, especially to avoid any reaction to a possible general cooling of the overall skin surface that might induce vasoconstriction.

Instrumentation

Instrumentation consisted of laser Doppler flow probes, thin ribbon or small bead type T thermocouples, and a kapton heat flux gauge mounted directly onto the skin at the site of active cooling. The area on the skin to which the cooling bladder was applied was covered with a thermal insulation barrier consisting of either a single loose layer of (ACE) elastic bandage or a (TED) stocking. The barrier also covered all thermal instrumentation placed directly onto the skin under the bladder but only surrounded the laser Doppler probes because they have a sensor elevation of about 1 cm above the skin. Thermocouples were placed on the surface of the cooling bladder at a location directly over one of the skin temperature measurements, and in the ice water bath. In some instances tee connectors with a thermocouple positioned in the side branch were inserted into inlet and outlet water lines adjacent to the bladder to monitor the water temperature at those points. Blood pressure was monitored intermittently via an arm cuff.

Skin blood flow was monitored with two laser Doppler flow systems: a MoorVMS-LDF2 two channel perfusion and temperature system and a MoorVMS-LDF1-HP single channel high power perfusion probe (Moor Instruments, Millwey, Axminster, Devon, UK). The LDF2 device supports two fiber optic probes that incorporate both laser Doppler and thermistor temperature measurement. The flow field sampling depth is relatively superficial, centered at about 0.5 mm into the skin, extending from near the surface to about 2 mm. The LDF1-HP device incorporates only a single perfusion probe, but with a higher power laser and a wider sensor separation resulting in a greater sampling depth centered at about 2 mm, extending from near the surface to about 4 mm. The probe heads were affixed to the skin via a flexible probe holder and a double-sided adhesive tape. The probe heads incorporate the fiber optic and sensor surface in a right angle configuration that stands about 1 cm proud of the skin surface. Consequently, when these probes were positioned underneath the cooling bladder, the cold source was held away from the skin for an area about 2 cm in diameter, resulting in temperatures somewhat higher than in regions where the bladder directly contacts the thermal barrier material.

Temperatures were monitored with ribbon or small (1 mm diameter) bead type T (copper/constantan) thermocouples (Omega Engineering, Stamford, Conn.). The ribbon sensors have a flat thermal element 25 μm thick that is embedded in a thin polyimide sheath that is taped to the skin surface peripheral to the sensing element. The typical time constant for the thermocouples is on the order of 0.1 s, which is much shorter that necessary to follow the temperature transients for cryotherapy experiments. The primary advantages of these sensors is their low mass and thin cross section that cause minimal perturbation to the temperature field, and their mechanical flexibility that allows them to conform to the surface contours of the skin. Multiple thermocouples, typically at least six, were affixed to the skin at locations beneath the cooling bladder as well as at proximal and/or distal sites. In addition, a heat flux gauge (Omega Engineering, Stamford, Conn.) was mounted to the skin beneath the cooling bladder to provide a continuous measure of the heat flow at the skin surface. The heat flux gauge was also fabricated in a thin polyimide (kapton) sheath, and it included an embedded type K (chromel/alumel) thermocouple. This paper does not report the heat flux data.

All electrical sensor outputs (fiber optic blood perfusion and temperature, thermocouples, heat flux gauge) were read into a data acquisition interface (DAQ) (NI 9172, 9174, 9201, 9205, 9211, and 9213; National Instruments, Austin, Tex.) and recorded using NI LabVIEW Signal Express software. Data was sampled at 12 Hz, and the thermocouple inputs were down-sampled to 3 Hz. Data files subsequently were transferred to a host computer where they were subjected to analysis and plotting using MATLAB (MathWorks, Natick, Mass.).

Periodic manual measurements of the subject blood pressure were performed using a sphygmomanometer and stethoscope. These values were applied to normalize the blood perfusion measurements to calculate values for cutaneous vascular conductance (CVC).

CVC=laser Doppler flux·100/mean arterial pressure

Approximately one hour was required to position and connect all of the instrumentation onto the subject at the beginning of an experimental session and to confirm its function. This process allowed the subject to become acclimated to the experimental environment. Baseline data acquisition was initiated at this point in time. Subsequently the cooling bladder was secured in place over the instrumentation and thermal insulation barrier. Each protocol consisted of an initial period of baseline data with no water flow through the bladder, followed by active cooling with flowing water, and then passive rewarming with no water flow. The cooling bladder remained in place on the subject throughout the entire test protocol.

Experimental Protocols

The room temperature water remained captured within the bladder until the cooling process was commenced by the pumping of ice water. After all of the instrumentation was in place and its function verified and a layer of insulating material was applied to the testing area, the cooling bladder was placed onto the treatment site, after which the bladder surface temperature rapidly approached that of the skin. Each protocol was initiated with a 10 to 45 minute period of baseline data acquisition before ice water flow into the bladder was initiated. The purpose of this period was to obtain values for perfusion and temperature to which subsequent changes elicited by the cryotherapy procedure could be normalized. Although the values of temperature and especially of blood perfusion were never perfectly static, baseline variations were small in comparison with those caused by the cooling procedures.

The cooling process was initiated at the end of baseline data collection by turning on the pump to produce a steady flow of ice water through the bladder (or increasing the elevation of the gravity feed device.) The duration of the cooling period was a primary control parameter evaluated in this study. Ice water flow through the bladder was maintained continuously for a wide range of times, including approximately 1, 2, 3, 5, 10, 30, 40, 60, and 80 minutes, as well as others, and in some trials, the protocol was repeated one or more times.

The cooling process was terminated by turning off the ice water pump, following which both the bladder and the underlying tissues began to warm via a combination of parasitic heat gain from the environment and active warming from within the tissues from metabolism and convection of blood. Both of the latter effects were depressed at the lower local tissue temperatures. The period of time over which the rewarming process was monitored also was varied over a wide range of values from 30 to 240 minutes. In some trials cooling was reinitiated following a defined period of rewarming to establish a cooling and rewarming cycle that could be reiterated.

One concern during data acquisition was whether pressurizing the cooling bladder with ice water would cause an artifactual signal in the underlying laser Doppler velocimeter probes that are exquisitely sensitive to relative movement with respect to the skin surface on which they are mounted. There frequently, but not always, are spikes in the perfusion signal in conjunction with initiating and stopping the flow of pressurized water through cooling bladders that are not of a physiological origin. Control data was obtained by pumping thermally neutral water at skin temperature through the bladder to determine whether the mechanical pressure owing to water flow caused an effect on the apparent blood perfusion measurements. All parameters for the control trials with the exception of the water temperature were the same as for the cryotherapy protocols.

Results

The reported experiments were conducted on healthy adult subjects covering the age range of 20-69 years. In total more than 100 trials have been conducted on 12 subjects. The selected results reported herein are representative of and consistent with the physiological response to the application of cryotherapy observed and measured in all trials.

The data are presented primarily in terms of plots of the time variation in temperature and blood perfusion at specific sites under the cooling bladder. For trials lasting as long as six hours and involving data acquired from 10 or more sensors at 1 Hz or greater, the amount of information generated is large, and it can only be reported selectively. Only certain sensor outputs are shown on individual plots for the sake of clarity. As would be anticipated, data are not replicated exactly among the various trials or even among various sensor locations for an individual trial, but a number of very important consistent behaviors have been identified. The results are presented in a format to emphasize the primary and most significant phenomena that have been measured and characterized. Perfusion data plots are the result of time averaging over a 3 minute moving window unless otherwise specified.

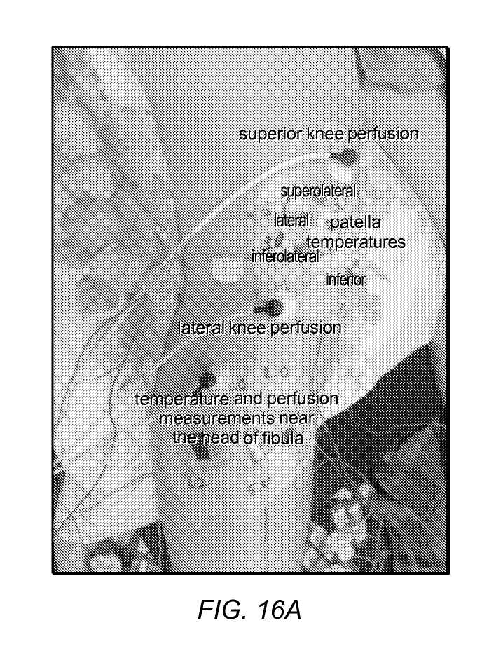

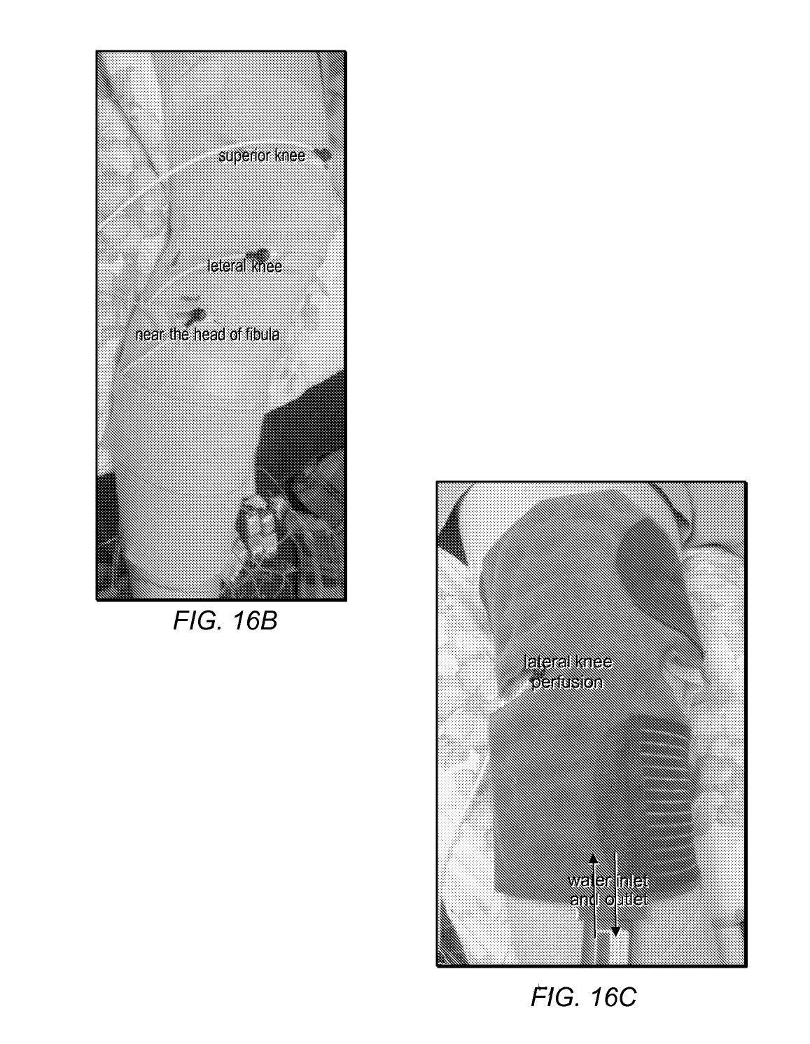

FIGS. 16A-B depict photographs of instrumentation applied to the right knee under the area to be covered by the cooling bladder. The view is from the ventral aspect. FIG. 16A shows sensors applied to the skin, including six smaller rectangular thermocouples, one larger square heat flux gauge, and three fiber optic probes for laser Doppler measurements. Numbers written on mounting tape indicate connection junctions of lead wires to the DAQ. FIG. 16B shows the same area after a thermal insulation barrier consisting of a single layer of ACE bandage wrap has been applied with no elastic stretching. FIG. 16C a Breg Polar Care cooling bladder positioned over the thermal barrier.

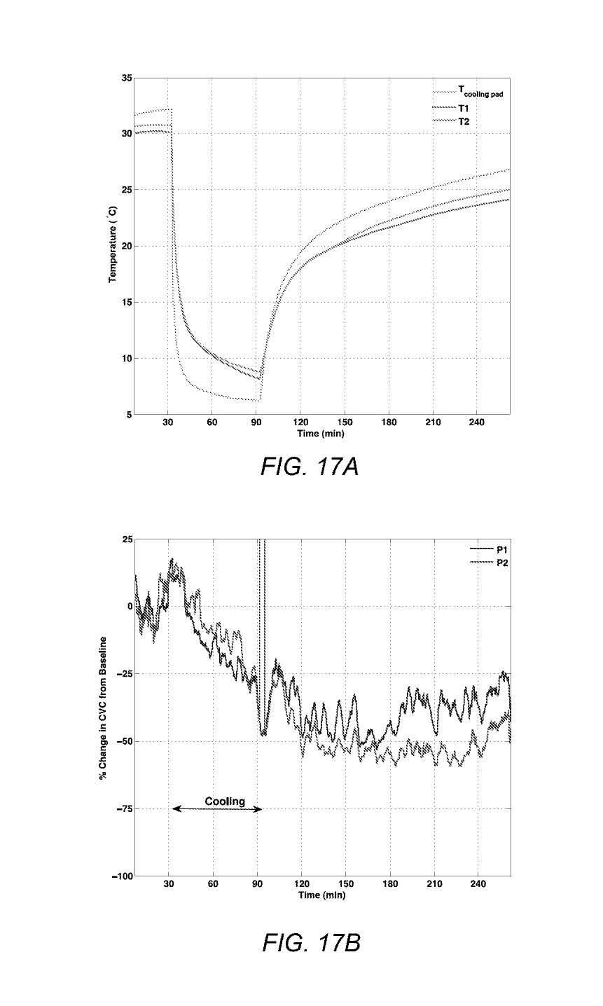

FIGS. 17A-B depict data for a cryotherapy trial with a DonJoy Iceman 1100 system applied to the shin consisting of 30 minutes of baseline data, followed by active cooling for 60 minutes and passive rewarming for 150 minutes. FIG. 17A depict temperature histories measured at two locations on the skin under the water perfusion bladder (red and green) and on the surface of the bladder (cyan). FIG. 17B shows P1 and P2 represent perfusion histories under the cooling bladder.

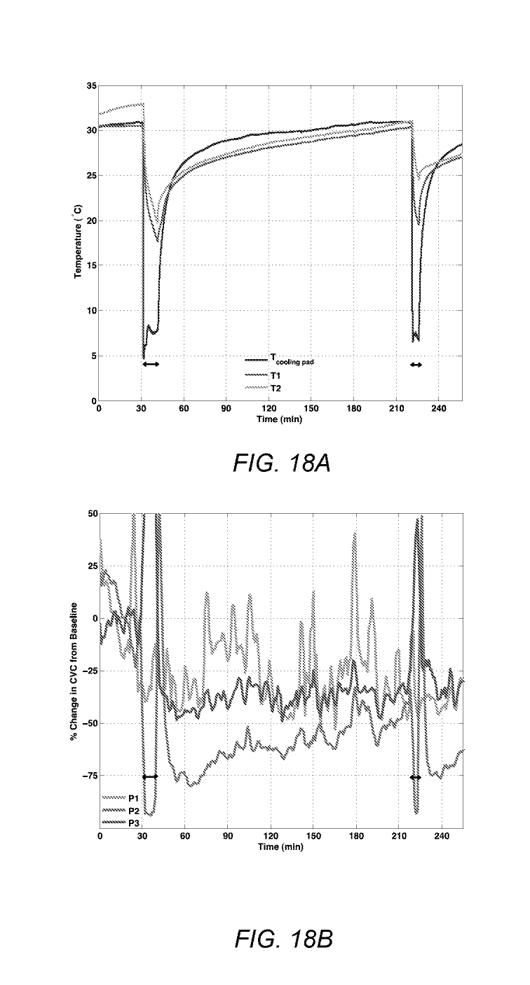

FIGS. 18A-B depict data for a cryotherapy trial with a Breg Polar Care 500 Lite system applied to the knee consisting of 30 minutes of baseline data, followed by active cooling for 10 minutes, and passive rewarming for 180 minutes, resumption of active cooling for 5 minutes, and passive rewarming for 20 minutes. Double arrows mark the duration of each cooling cycle. FIG. 18A depict temperature histories measured at two locations on the skin under the water perfusion bladder (red and green) and on the surface of the bladder (blue). FIG. 18B depicts two superficial (magenta and green) and one deep (red) perfusion histories under the cooling bladder.

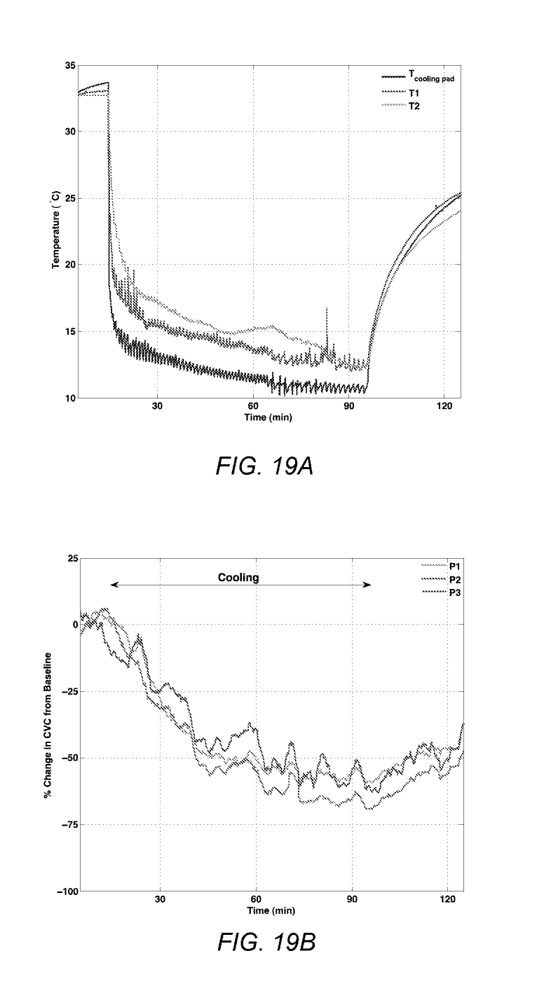

FIGS. 19A-B depict data for a cryotherapy trial with a DeRoyal T600 applied to the shoulder consisting of 15 minutes of baseline data, followed by active cooling for 80 minutes, and passive rewarming for 30 minutes. FIG. 19A depicts temperature histories measured at two locations on the skin under the water perfusion bladder (red and green) and on the surface of the bladder (blue). FIG. 19B depicts two superficial (magenta and green) and one deep (red) perfusion histories under the cooling bladder.

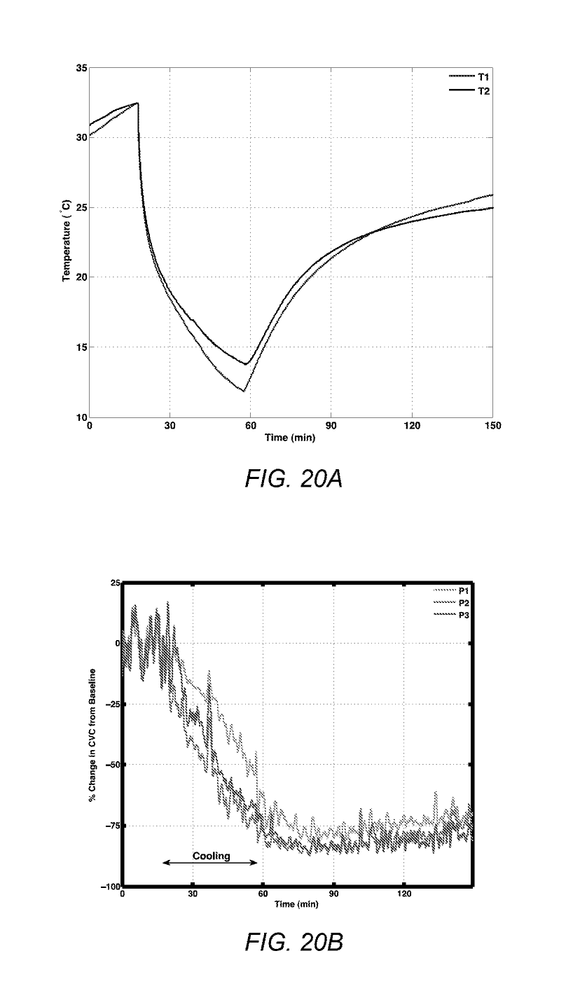

FIGS. 20A-B depict data for a cryotherapy trial with an Arctic Ice System applied to the calf and shin area of the lower leg, consisting of 15 minutes of baseline data, followed by active cooling for 40 minutes and passive rewarming for 90 minutes. FIG. 20A depict temperature histories measured at two locations on the skin surface under the cooling bladder (black and blue). FIG. 20B depict superficial (green and magenta) and deep (red) perfusion histories at three sites in the skin under the cooling bladder. Perfusion data was time averaged over a 1 minute moving window.

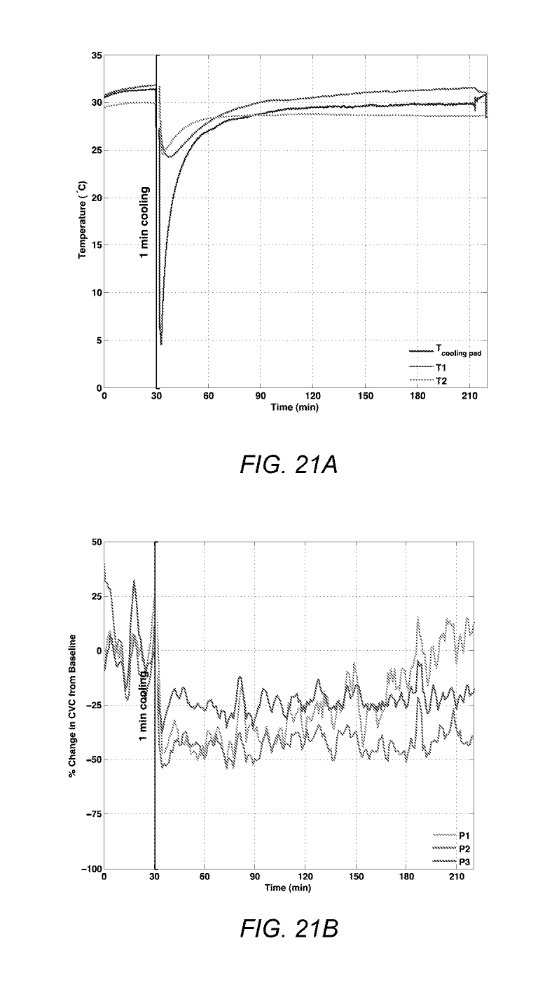

FIGS. 21A-B depict data for a cryotherapy trial with an Arctic Ice System applied to the calf and shin area of the lower leg, consisting of 30 minutes of baseline data, followed by active cooling for 1 minute and passive rewarming for 195 minutes. FIG. 21A depicts temperature histories measured at two locations on the skin surface under the cooling bladder (red and green) and on the surface of the bladder (blue). FIG. 21B depicts superficial (green and magenta) and deep (red) perfusion histories at three sites in the skin under the cooling bladder.

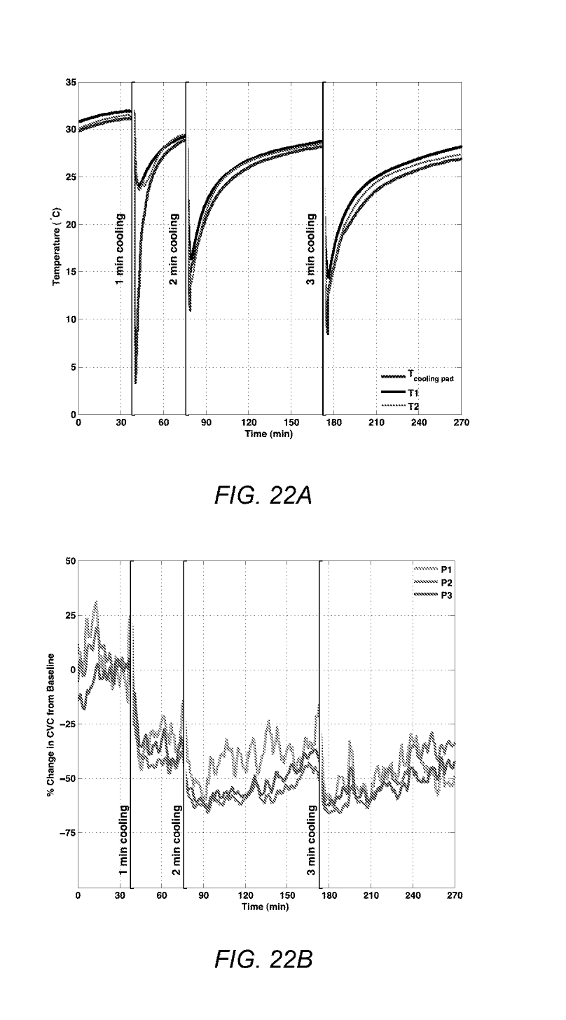

FIGS. 22A-B depict data for a cryotherapy trial with an Arctic Ice System applied to the calf area of the lower leg, consisting of 39 minutes of baseline data, followed in sequence by active cooling for 1 minute and passive rewarming for 37 minutes, 2 minutes of active cooling and 96 minutes of passive rewarming, and 3 minutes of active cooling and 122 minutes of passive rewarming FIG. 22A depicts temperature histories measured at two locations on the skin surface under the cooling bladder (red and black) and on the surface of the bladder (blue). FIG. 22B depicts superficial (green and magenta) and deep (red) perfusion histories at three site in the skin under the cooling bladder.

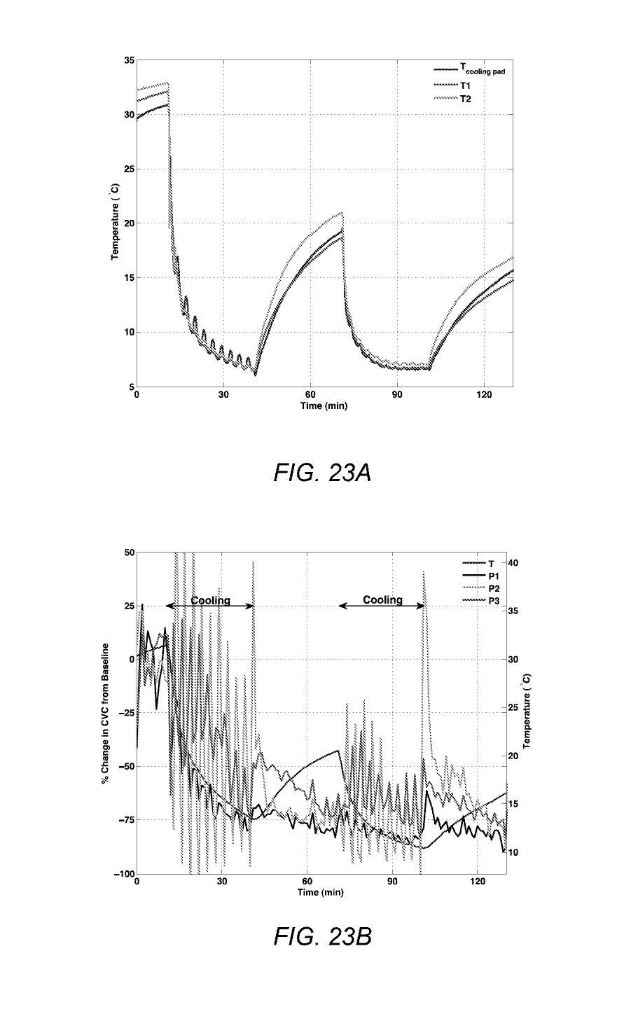

FIGS. 23A-B depict data for a trial with a Game Ready cryotherapy unit applied to the knee, consisting of 10 minutes of baseline data, followed sequentially by active cooling for 30 minutes and passive rewarming for 30 minutes, then a second identical episode of active cooling for 30 minutes and passive rewarming for 30 minutes. FIG. 23A depict temperature histories measured at two locations on the skin surface under the cooling bladder (red and green) and on the surface of the bladder (blue). FIG. 23B depict superficial (green and black) and deep (red) perfusion histories at three sites in the skin under the cooling bladder. The blue plot shows the temperature history measured via one laser Doppler probe. Note that the intermittent pressurization feature was operative during this trial in which an outer air bladder was periodically inflated and deflated for 90 seconds sequentially.

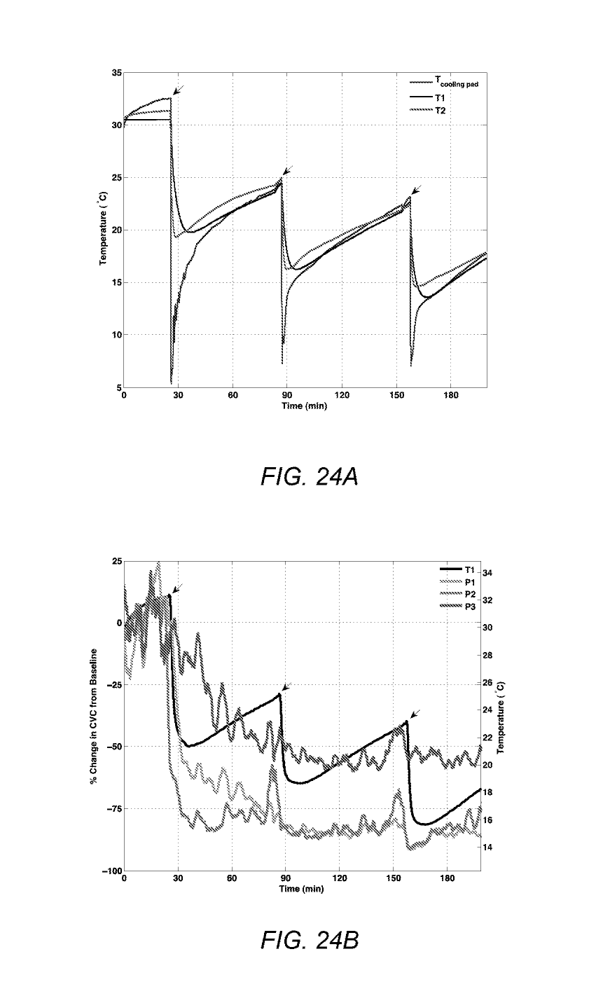

FIGS. 24A-B depict data for a trial with an Aircast Cryo/Cuff cryotherapy unit applied to the knee, consisting of 26 minutes of baseline data, followed which the cooling bladder was filled with ice water by gravity feed with the source volume elevated approximately 0.3 m above the bladder and kept in position with the air vent opened for approximately 56 minutes. The source volume was then lowered below the bladder to allow the water to drain from the bladder, and at 60 elapsed minutes after the first cooling episode it was again raised 0.3 m above the bladder to provide a second cooling cycle. A third cooling episode was initiated after 72 elapsed minutes. A slight perturbation to the slope of the temperature plots can be observed during the lowering of the source volumes just prior to the start of subsequent cooling episodes. FIG. 24A depicts temperature histories measured at two locations on the skin surface under the cooling bladder (red and black) and on the surface of the bladder (blue). FIG. 24B depicts superficial (green and magenta) and deep (red) perfusion histories at three sites in the skin under the cooling bladder. The black plot shows the temperature histories measured via the thermistor on one of the two low power laser Doppler probes. Black arrows point to the start of each cooling period.

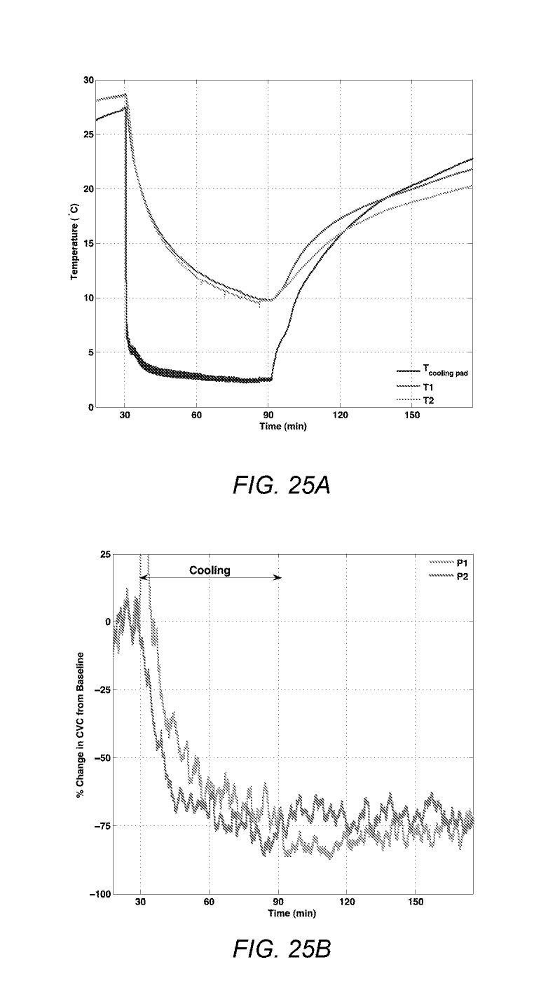

FIGS. 25A-B depict data for a trial with a Breg Polar Care 300 cryotherapy unit applied to the knee, consisting of 30 minutes of baseline data, followed by active cooling for 60 minutes and passive rewarming for 84 minutes. FIG. 25A depict temperature histories measured at two locations on the skin surface under the cooling bladder (red and green) and on the surface of the bladder (blue). FIG. 25B depict superficial (green and magenta) perfusion histories at two sites in the skin under the cooling bladder.

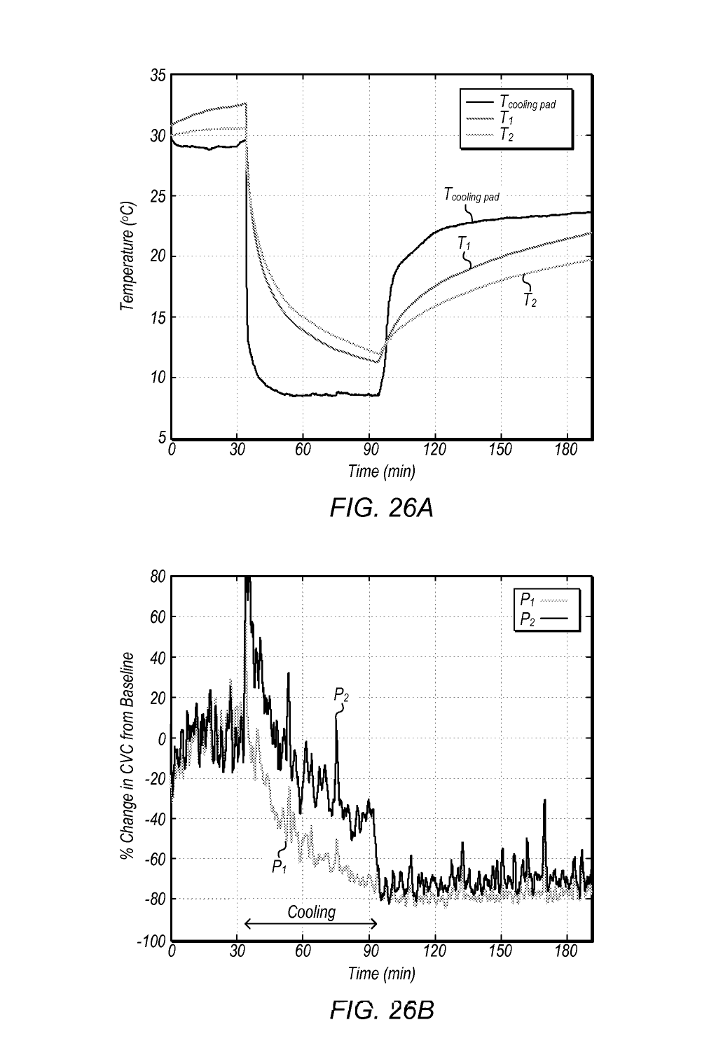

FIGS. 26A-B depict data for a trial with a DonJoy Iceman 1100 unit applied to the ankle, including 34 minutes of baseline data, followed by active cooling for 60 minutes and passive rewarming for 86 minutes. FIG. 26A depict temperature histories measured at two locations on the skin surface under the cooling bladder (red and green) and on the surface of the bladder (blue). FIG. 26B depict skin blood perfusion measured at two locations under the cooling bladder. Magenta perfusion plot shows a sudden increase and decrease with the initiation and termination of water flow through the cooling bladder, respectively, due to sudden change in applied mechanical pressure. This effect is not seen in the green perfusion plot. This variation in response could be due to differences in the anatomical locations of the probes relative to the water flow pattern within the bladder. Double arrows mark the duration of cooling which lasted for 60 minutes.

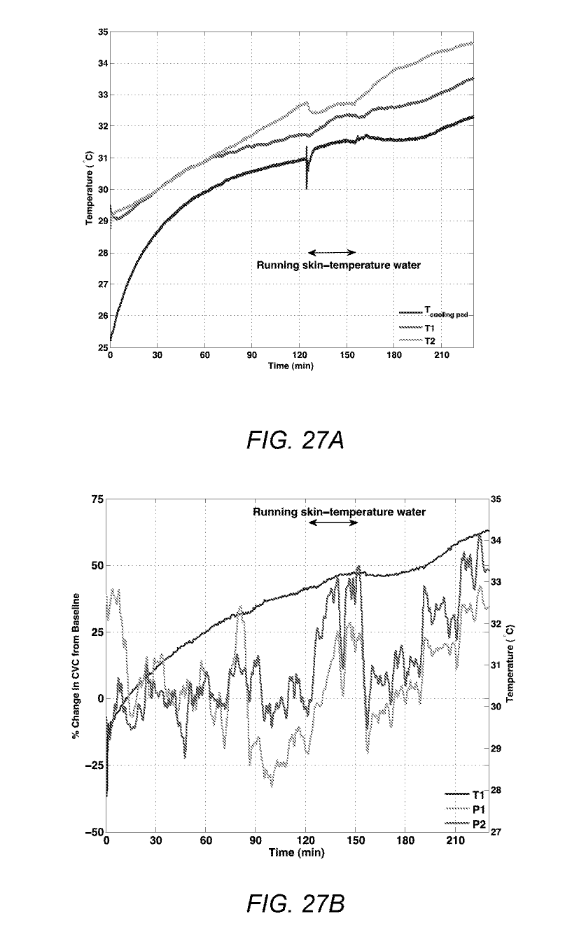

FIGS. 27A-B depict a control experiment with full instrumentation and the Breg Polar Care 500 Lite cooling bladder applied to the knee. Thermally neutral skin temperature water at about 32° C. was pumped through the bladder from 125 to 155 minutes. There was no flow through the bladder at other times. FIG. 27A depicts temperature histories measured at two sites on the skin under the cooling bladder (red and green) and on the surface of the bladder (blue). FIG. 27B depicts skin blood perfusion measured at two locations under the cooling bladder. P1 (green) and P2 (red) represent superficial and deep perfusion measurements, respectively. Also, the temperature measured via the thermistor on the laser Doppler probe at the superficial perfusion site is plotted (blue).

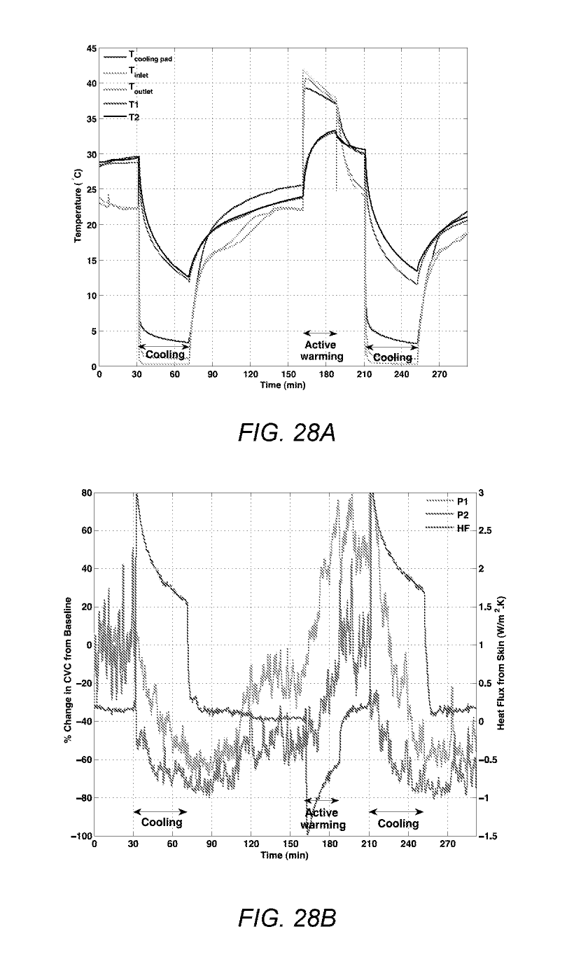

FIGS. 28A-B depict data for a trial with a Breg Polar Care 500 cryotherapy unit applied to the knee, consisting of 30 minutes of baseline data, followed by active cooling for 40 minutes and passive rewarming for 90 minutes, then active rewarming for 27 minutes, passive heat transfer for 22 minutes, then active cooling for 42 minutes and passive rewarming for 40 minutes. FIG. 28A depicts temperature histories measured at two locations on the skin surface under the cooling bladder (red and green), on the surface of the bladder (blue), and in the water flow lines at the bladder inlet (color) and outlet (color). FIG. 28B depicts superficial (green and magenta) perfusion histories at two sites in the skin under the cooling bladder. The temperatures measured via the thermistors on both laser Doppler probes are plotted to show the correlation between perfusion and temperature.

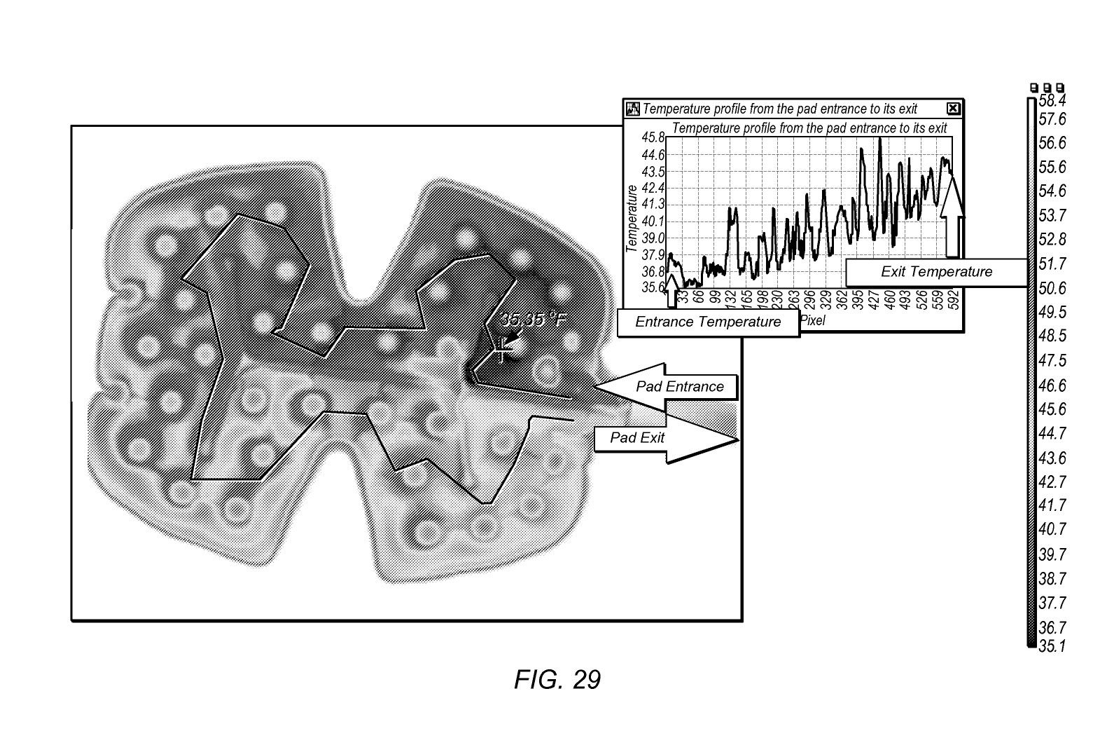

FIG. 29 depicts an infrared thermograph of the temperature distribution on the surface of a Breg Polar Care 300 knee pad during perfusion with ice water. The surface was exposed to room air at 23° C. The system was operated for a sufficient time to reach steady state. There was no water condensation on the pad surface. Temperatures are indicated on the pseudocolor scale. An exemplar flow pathway was manually traced between the inlet and outlet as indicated by the red line, with the temperatures of individual image pixels along the path plotted to the upper right. The warming differential between inlet and outlet is about 4.5° C. Areas near the periphery of the flow and where the upper and lower surfaces of the bladder are welded together are substantially warmer. Temperature differences over the bladder surface are as large as 8° C.

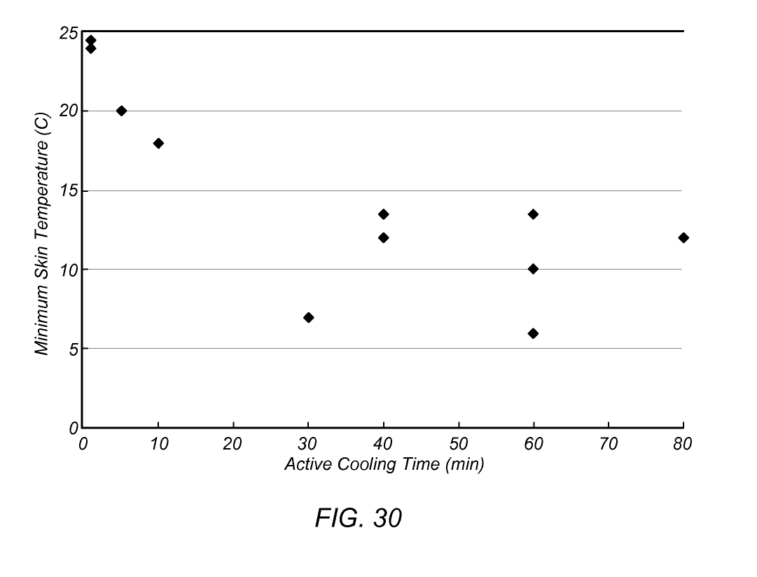

FIG. 30 depicts the minimum temperature achieved on the skin surface as a function of the duration of the initial episode of cooling applied with a cryotherapy cooling bladder with continuously circulating ice water.

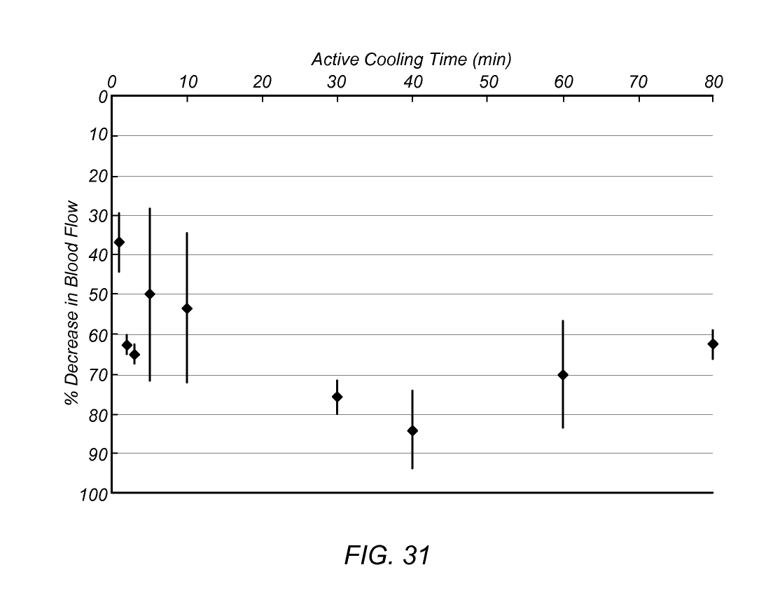

FIG. 31 depicts a decrease in cutaneous blood perfusion as a function of the period of application of continuous flow ice water cryotherapy. Average and standard deviation values are based on data from each individual perfusion measurement from all trials. n varies between 2 and 6 as can be observed from FIGS. 17-23, 25-26, and 27.

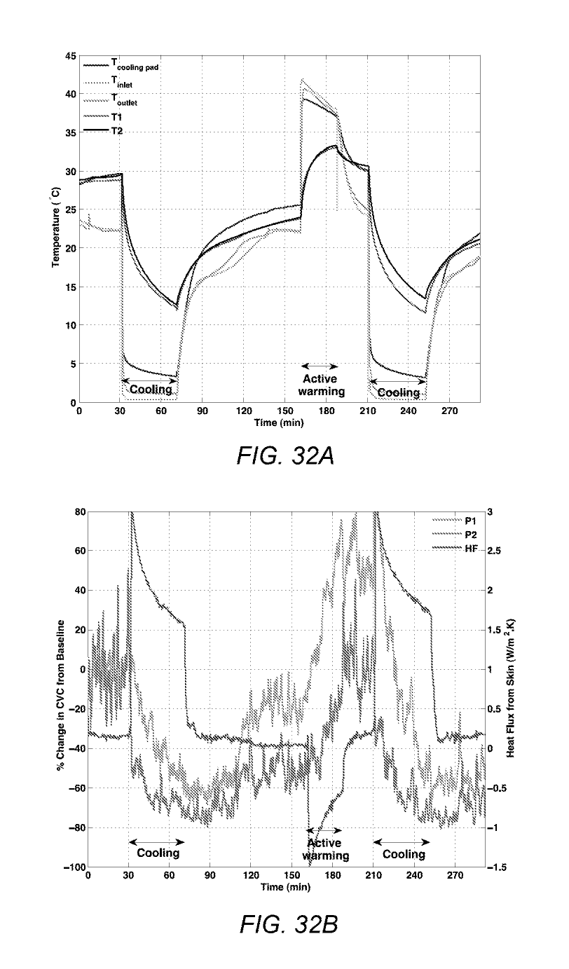

FIGS. 32A-B depict data for a trial with a Breg Polar Care 500 cryotherapy unit applied to the knee, consisting of 30 minutes of baseline data, followed by active cooling for 40 minutes and passive rewarming for 90 minutes, then active rewarming for 27 minutes, passive heat transfer for 22 minutes, then active cooling for 42 minutes and passive rewarming for 40 minutes. FIG. 32A depicts temperature histories measured at two locations on the skin surface under the cooling bladder (red and green), on the surface of the bladder (blue), and in the water flow lines at the bladder inlet (color) and outlet (color). FIG. 32B depicts superficial (green and magenta) perfusion histories at two sites in the skin under the cooling bladder. The temperatures measured via the thermistors on both laser Doppler probes are plotted to show the correlation between perfusion and temperature.

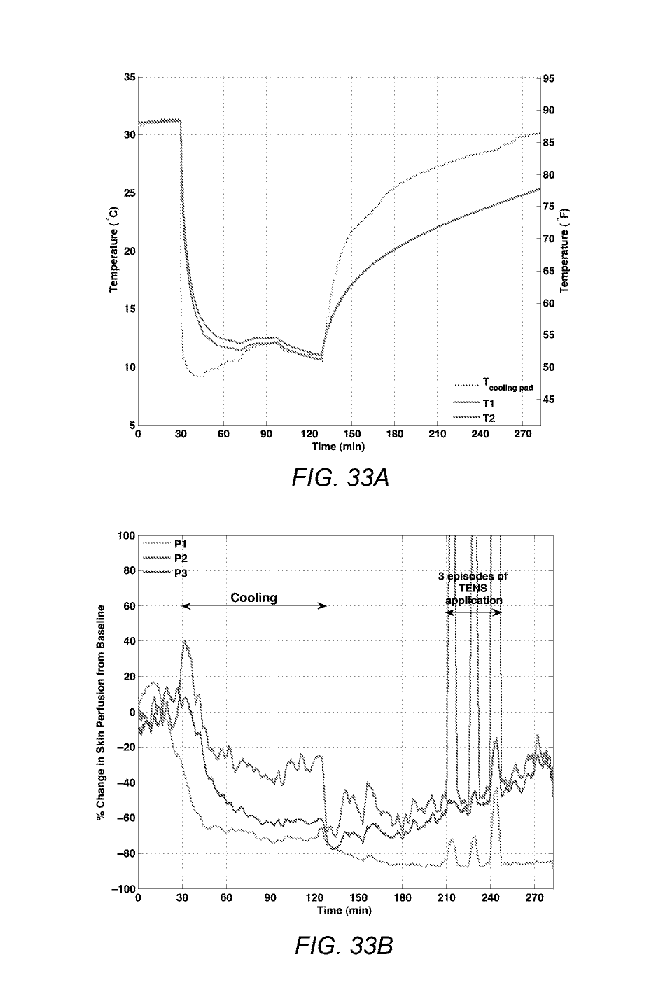

FIGS. 33A-B depict data for a trial with a DonJoy Iceman cryotherapy unit applied to the knee. The thermal protocol consisted of 30 minutes of baseline cooling, followed by 100 minutes of active cooling, and finally 80 minutes of passive rewarming Between 210 and 255 minutes there were three episodes of transcutaneous electrical nerve stimulation (TENS) applied for 5 minutes each with a 15 minute resting inbetween. FIG. 33A depict temperature histories measured at two skin locations (black and magenta) and on the cooling pad (blue). FIG. 33B depict superficial (green and magenta) and deep (red) perfusion histories at three sites in the skin under the cooling bladder.

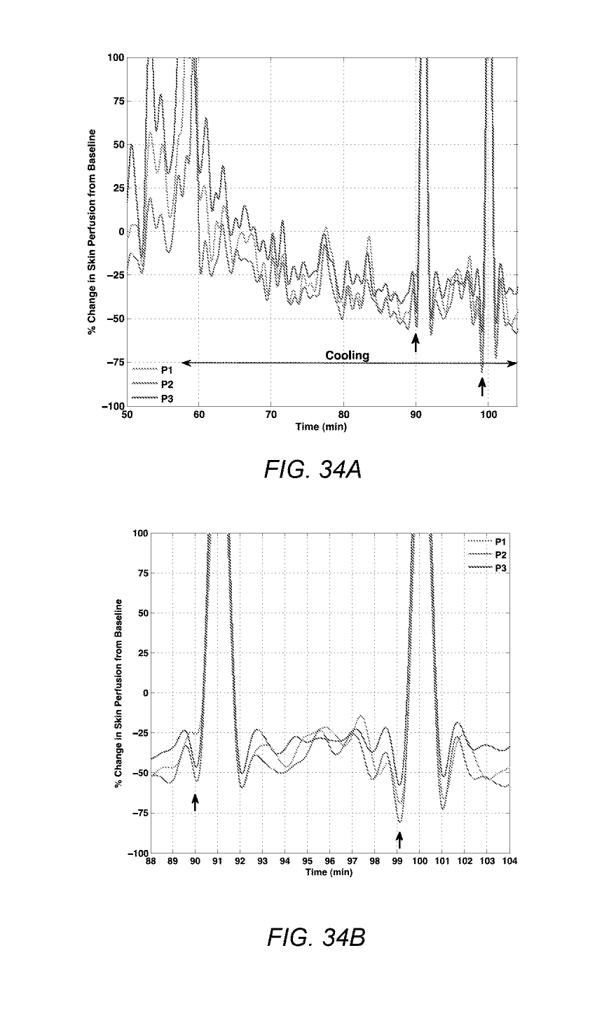

FIGS. 34A-B depict data for a trial with a DonJoy Iceman cryotherapy unit applied to the knee. The thermal protocol consisted of 58 minutes of baseline data, followed by 50 minutes of active cooling, and finally 60 minutes of passive rewarming. Two episodes of machine driven ankle flexion of two minutes duration were imposed during active cooling. Only perfusion data during an intermediate portion of the protocol timeline is shown. FIG. 34A depicts superficial (green and magenta) and deep (red) perfusion histories at three sites in the skin under the cooling bladder. FIG. 34B depict an expanded time scale during two ankle flexion episodes starting at 90 and 99 minutes during active cooling.

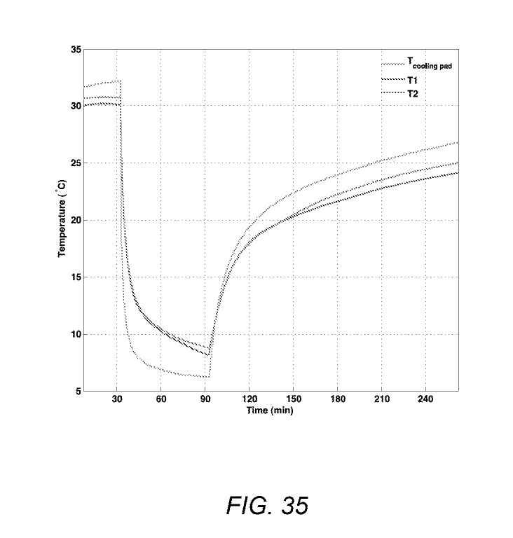

FIG. 35 depicts data for a trial with a DonJoy Iceman cryotherapy unit applied to the knee. The thermal protocol consisted of 30 minutes of baseline data, followed by 60 minutes of active cooling, and finally 180 minutes of passive rewarming A Covidien Kendall Novamedix A-V Impulse System Model 6060 blood pump was used concurrently on the foot of the leg receiving cryotherapy. Temperature histories at two sites on the skin under the cooling pad (green and magenta) and on the surface of the pad (blue) are shown.

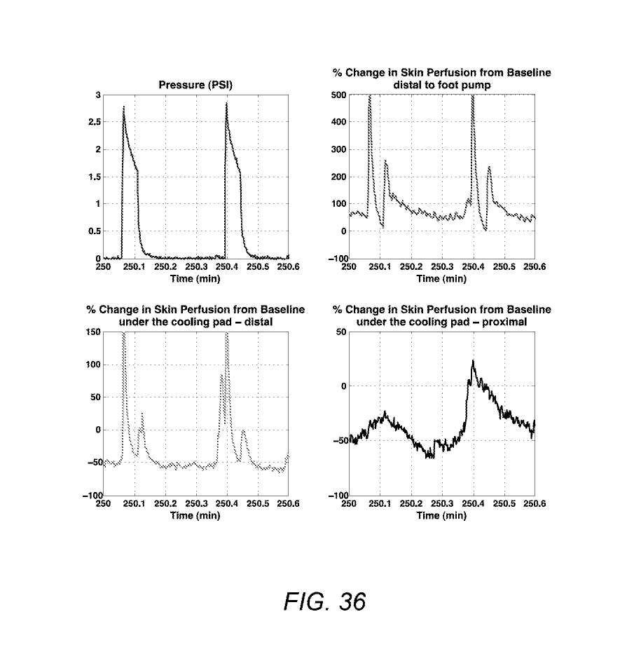

FIG. 36 depicts effect of the operation of the impulse blood pump for two cycles of operation while the circulation was in a state of cryotherapy-induced vasoconstriction. The upper left graph (blue) shows the time variation of pressure within the pneumatic foot cuff that causes intermittent blood flow increases. The pressurization period is approximately 3.5 seconds following a rise to maximum pressure of less than 0.5 second. The pressure impulses were programmed to occur at 21 second intervals. The upper right graph (red) shows transients in blood flow measured distally of the foot on the great toe. There is a biphasic response to the pressure impulse in the cuff: the first increase in flow is associated with deoxygenated blood being forced from the plantar arteriovenous anastomoses; the second increase is due to the refill of the vasculature with fresh oxygenated after pressure is released from the cuff. The lower left graph (green) shows blood flow measured at the distal end of the cryotherapy pad, closest to the blood pump. The same biphasic blood flow increase was measured at this location, for the foregoing reasons. The lower right graph (black) shows blood flow measured at the proximal end of the cryotherapy pad, furthest from the pump.

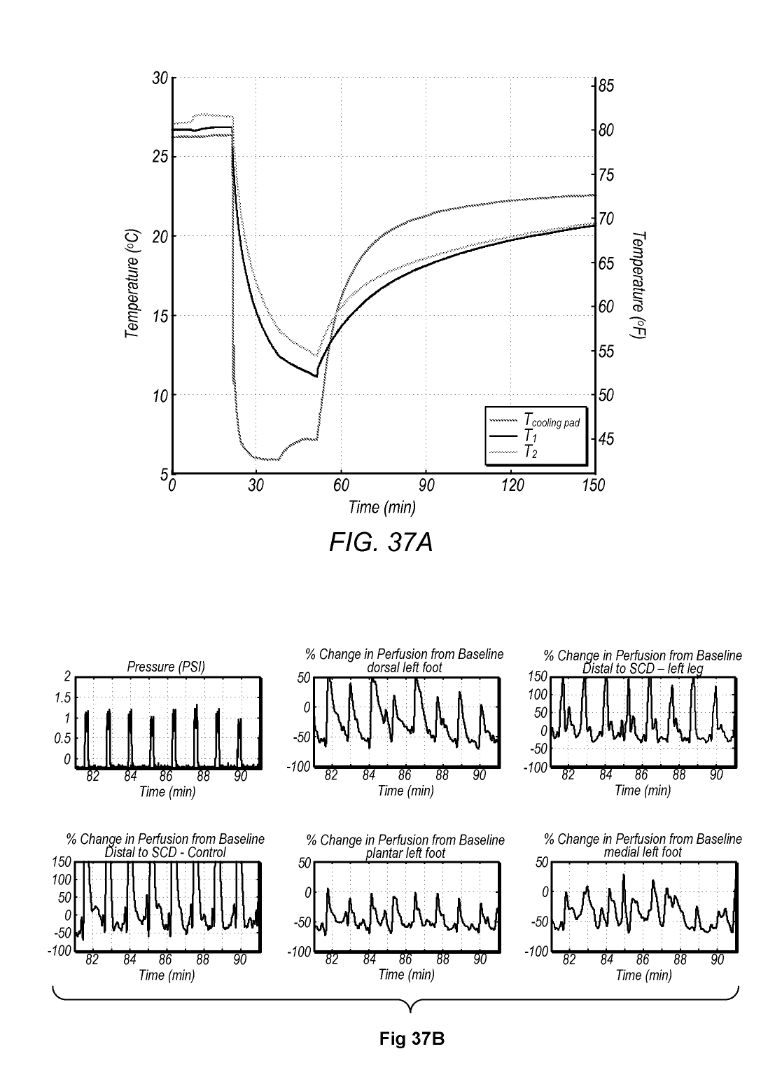

FIGS. 37A-B depict data for a trial with a DonJoy Iceman cryotherapy unit applied to the ankle. The thermal protocol consisted of 20 minutes of baseline data, followed by 30 minutes of active cooling, and 100 minutes of passive rewarming A Covidien Kendall Model 6325 Sequential Compression Device (SCD) blood pump and associated compression sleeves was used concurrently on the lower leg below the knee and above the cryotherapy pad. FIG. 37A depicts temperature histories at two sites on the skin under the cooling pad (red and yellow) and on the surface of the pad (blue). FIG. 37B depicts plots of: air pressure history in the SCD (upper left, blue); blood flow measured at five locations in response to operation of the SCD, as indicated in the legends. The red, black and brown plots are under the cryotherapy pad. The magenta plot is a non-cooled area between the SCD and the pad. The green plot is control data for the opposite leg that had no cryotherapy pad or cooling.

The foregoing figures illustrate the existence of a profound and persistent state of ischemic following the cessation of active cooling when the cooling bladder and underlying tissues are allowed to passively rewarm via natural convection with ambient air. This general behavior has been observed for the entire spectrum of standard cryotherapy protocols we have investigated. In this context, we undertook a nonstandard protocol in which an active rewarming process was imposed following a period of usual passive rewarming by circulating heated water through the cooling bladder. The prime objective of this study was to see if active heating could be used to break the state of post-cooling ischemia. Following active rewarming, a second cycle of active cooling and passive rewarming was applied to determine whether the state of persistent post cooling ischemia would be reestablished. The transient temperature and perfusion plots for this study are shown in FIG. 28.

Inspection of the thermal data presented in the foregoing figures shows that there is variation in the temperatures measured at different skin sites under a cooling bladder during individual trials. One possible source of these variations may be due to thermal gradients on the surface of the cooling bladder. Therefore, we acquired thermal images of the surface of a cooling bladder through which water was circulating to quantify the spatial disparities in the temperature pattern. For this purpose we placed a bladder in an unfolded configuration onto a flat surface with the cooling surface oriented upwards. We positioned a thermal camera (FLIR Systems, Boston, Mass.) directly above the bladder to record the two dimensional pattern of temperature on the surface. An example image for a Breg Polar Care 300 knee pad is shown in FIG. 29.

We have also inserted a mobile thermocouple on the skin surface near the geometric center of the pad beneath the thermal barrier during a cryotherapy protocol and pulled it toward the edge during the active cooling phase to establish the temperature field. Similar large temperature gradients were measured, on the order of 6-8° C. in the areas that were tested. There clearly are significant temperature gradients created by the thermal pattern on the surface of the water perfusion bladder, and these will cause similar thermal gradient patterns on the skin.

The data plots, as an aggregate, document temperature and blood perfusion characteristics that are common across all of the trials on various devices and conducted on different areas of the body and that provide broad insights into the physiological response to cryotherapy. Perhaps the most significant outcome of this study is that the application of a cryotherapy device creates a state of persistent ischemia at the treatment site. The implication of this outcome is that exposure to ischemia over an extended period of time may contribute to the development of tissue necrosis and neuropathy, as has been recognized widely in prior literature. There are also differences among the measured temperatures and blood perfusions between and within the various trials that are important to recognize and understand. These are discussed as follows.

### Temperature Effects

As would be anticipated, during cooling the temperature on the bladder surface drops ahead of the skin temperature, creating a gradient in which heat flows from the tissue to the bladder and the cold water circulating through it. For trials in which active cooling lasted long enough for the bladder surface temperature to reach a steady state value (FIGS. 19A, 25A, 26A, 28A), the skin continued to cool long after the bladder was at steady state. This behavior is due in large part to the much larger thermal mass of the tissue being cooled in comparison with the bladder and to the fact that the bladder is located more proximally to the heat sink provided by the circulating ice water. In like manner, in FIG. 28A the temperature of the water circulating in the bladder precedes the bladder surface temperature in decreasing during cooling starting at 30 and 210 minutes and in increasing during warming starting at 160 minutes. The difference between the inlet and outlet temperatures is less than 1° C. This temperature difference, multiplied by the specific heat of water and the mass flow rate through the bladder, provides a direct measure of the rate of heat transfer between the bladder and the skin.

Another feature of the temperature data is that the rates of temperature drop during active cooling are always much larger than the rates of temperature rise during passive rewarming. This phenomenon is a result of cooling being driven by forced convection with water interior to the bladder, whereas warming is driven by natural convection with air exterior to the bladder. Comparison of the material properties (thermal conductivity, specific heat, density) of water and air and of the relative efficacy of forced and natural convection both point to heat transfer during cooling being more efficient than during warming Plus, during rewarming heat must be conducted through an additional layer of insulation at the outer layer of the bladder. Since the bladder is closer to the environmental heating source during warming than is the underlying tissue, the bladder surface temperature leads the skin temperatures during the warming process. The differential in heat transfer rates during active and passive rewarming processes is illustrated dramatically in FIG. 28A in which the slopes of the tissue temperature curves increased abruptly when warm water was perfused through the cryotherapy bladder. However, the higher environmental temperature also caused a greater heat transfer, which is a confounding factor in interpreting the slopes of the temperature profiles. At 187 minutes when the water perfusion through the bladder was ceased, converting the environment for the skin from forced to free convection, albeit with the same water temperature in the bladder, there is an immediate change from an increasing to a decreasing slope of the transient temperature gradient. This inversion of the slopes of the skin temperature curves illustrates clearly the strong effect imposed on the underlying tissue by switching from a forced to a free convection boundary condition. Operation of the bladder with forced convection flow of water is an important component of the functionality of cryotherapy units. Another example of this phenomenon is seen by comparing the cooling curves for the Aircast Cryo/Cuff device in FIG. 24A with those of the forced convection devices in all other cryotherapy trial figures. The Cryo/Cuff device operates via a single admission of ice water into the bladder where it remains static for the duration of the cooling cycle in the absence of a forced convection effect that is able to sustain a continuous high rate of heat removal from the tissue. Therefore, following an initial drop in temperature on the surface of the bladder, there is a continuous rise in temperature as the bladder absorbs heat from the underlying tissue. The rate of heat transfer decreases continuously as the temperature differential between the bladder and the skin diminishes.

In all of the trials the temperatures of the skin and the bladder were rising during the initial baseline period during which there is no active temperature control imposed. This trend occurs because metabolism and blood flow in the tissue provide an inner source of heat, and the outer bladder layer provides insulation of underlying structures. The result is that energy is generated and transported within the tissue at a rate larger than it can be lost via passive heat transfer to the environment, resulting in a net accumulation of energy that drives the temperatures upward prior to starting the circulation of ice water through the bladder.

The data in all of the trials also show that there were always positional differences in temperature histories. For example, there may be points on the skin surface with temperatures as low as 8-9° C., whereas on occasions during a single trial there may be other locations that simultaneously are 20° C. or higher. For clarity in viewing and interpreting the temperature data we have presented plots for only a minimal number of measurement sites, but much more data was gathered that supports this behavior. There are multiple potential contributing causes for spatial variations in temperature. (i) There may be differences in the local thicknesses of the thermal barrier. Typically a thermal barrier is placed between the bladder surface and the skin, although in general the device manufacturers do not define or specify specific standards for the thermal properties of barriers provided by end users. This effect is most probable when the barrier is applied as a wrap that may have a nonuniform pattern of application causing uneven thermal resistance. (ii) The temperature distribution on the surface of the cooling bladder is known to have spatial variations, as shown in FIG. 29. These variations will translate to gradients on the skin surface. (iii) The bladder may not fit uniformly against the thermal barrier and the underlying tissue, resulting in creation of insulating air gaps that will act as a thermal contact resistance, depending on the relative geometries of the bladder and the anatomy, causing a larger temperature drop between the skin and bladder. This effect is difficult to quantify and predict, but it is very real. We have observed at sites of poor fit a skin to bladder temperature differential that can be 15-20° C. larger than at locations where there is an intimate fit. The local physiological response will be modulated if the conformation of the cooling bladder to the body surface is poor. (iv) There may be areas of higher contact pressure between the bladder and the thermal barrier and underlying tissue. Greater pressure will reduce contact thermal resistance and may also compress the barrier material, lowering its resistance. The thermal effect of increased applied pressure on reducing the heat flow resistance is seen clearly in FIG. 23A for operation of the Game Ready device with cyclic pressure applied to the pneumatic cuff. Increased pressure reduces the temperature drop between the bladder and skin surface.

The cryotherapy trials all involve the circulation of water from an ice bath through a bladder placed on the subject at a simulated treatment site. The water flow tubes are all insulated with a light foam material to minimize heat gain from the surrounding air. Some cryotherapy systems offer a control of the water temperature as it is supplied to the cooling bladder. For systems in which ice water is pumped directly to the bladder, in line measurements of water temperature as it enters the bladder show values as low as 0.5° C. As steady operating conditions are approached, the temperature of the outer cooling surface of the bladder may be as low as 3-6° C., depending on the thermal resistance of the bladder material and the effectiveness of convection heat transfer with the water as it flows through the bladder.

The therapeutic (and possibly injurious) response elicited in tissue occurs as a function of the applied temperature and duration on the skin surface. Thus, variations in the temperature pattern imposed onto the surface of tissue may compromise the execution of cryotherapy, resulting in an unintended or unwanted response. Accurately producing a targeted temperature field onto a treatment area remains a fundamental challenge in perfecting cryotherapy devices.

### Blood Perfusion Effects

The predominate feature of the blood perfusion data is that there is consistently a large hysteresis between the transient plots of blood flow and temperature. The falling temperature affected by activation of the cryotherapy device causes a drop in blood perfusion. This effect is well known and is one of the principle mechanisms of cryotherapy in controlling the inflammatory and swelling process. However, after active cooling is terminated and the skin temperature rises, the level of blood perfusion does not follow as it does during cooling. The result is a state of persistent ischemia that remains long after any active application of cryotherapy has been terminated. As noted earlier, we have now conducted more than 100 instrumented cryotherapy trials, all on healthy adult subjects, and every trial demonstrated this behavior. In the absence of instrumentation to monitor blood perfusion, as is commonly the case for the clinical use of cryotherapy devices, this phenomenon is not likely to be detected or to be perceived. Nonetheless, the affected tissue area will be deprived of a normal supply of oxygenated blood with nutrients, and toxic metabolic byproducts will accumulate locally. The result is a condition that can lead to cell injury and death which, if enabled to accrue on a large scale, may be manifested as clinically observed necrosis and/or neuropathy, and the more general descriptor of nonfreezing cold injury. As noted in the background section, the literature of cryotherapy has many citations of these types of outcomes from cryotherapy.

There are a number of specific features of the blood perfusion response to cryotherapy in addition to persistent ischemia that may be addressed. It is readily apparent that the blood perfusion curves are not as smooth as are the temperature curves, which is due to the differential in the nature of the control mechanisms. Regulation of the human cardiovascular system has been studied and written about extensively for many years. One of the more comprehensive and elegant reviews is found in the book of Rowell to which readers are referred for details. As Rowell notes, “The primary function of the cardiovascular system is to deliver oxygen and nutrients to the tissues and to remove their waste products . . . . Other important functions of the peripheral circulation are the regulation of blood pressure, body temperature, and distribution of blood volume, and the performance of the heart.” Simply stated, humans may experience variations in local blood flow in conjunction with changes in the driving pressure from the pumping heart and in the continuous redistribution of blood volume around the body via exquisite adjustments in vasomotor function that cause modulations in the local flow resistance. The latter factor is undoubtedly the primary source of the relatively rapid small scale fluctuations in local blood flow rates observed in all of the data reported herein. These fluctuations occur in the normal functioning of the cardiovascular system and are not a consequence of the application of cryotherapy, as evidenced by data acquired during the baseline periods.

The laser Doppler flow measurements are subject to artifact generation owing to changes in the geometry of the probe and the skin surface. Such changes can occur if the subject moves or even flexes a muscle in the measurement area. Therefore, the subjects were exercised to remain passive through experimental trials to the greatest extent possible. Another source of artifacts is the application or removal of a force that presses the probe normally or obliquely against the skin surface, as occurs when a cryotherapy unit pump establishes or discontinues the flow of ice water through a cooling bladder that overlies a probe. This effect can be observed in many, but not all, of the blood flow plots in conjunction with the start and end of the active cooling periods. The control study of FIG. 27 demonstrates the pressure induced perturbation in flow measurement in the absence of a thermal influence on blood perfusion. A similar effect is observed in the data presented in FIG. 26. In this experiment, two blood perfusion probes were applied to the skin under the cooling bladder. There are sudden changes in flow measurements with the start and stop of water flow in P2 (magenta line) whereas, P1 (green line) only experiences a transient increase in flow with the start of the cooling and without any similar response with the stop of the water flow. As long as water flow was maintained through the cooling bladder, there was a constant upward offset in the value for P2 that disappeared when the flow stopped. The magnitude of the offset was approximately equal at the beginning and the end of the flow period. The difference in response between the two probes is likely due to their relative positioning with respect to the bladder that could influence the degree of pressure change they experience during the flow of water.

A pressure effect on blood perfusion measurements is also quite apparent in FIG. 23B for operation of the Game Ready cryotherapy unit with the pneumatic pressurization cuff activated periodically during cooling. Three probes were positioned on the skin beneath the cooling bladder during this trial, one deep and two superficial. Although all three probes had flow measurements altered by the application and release of pressure, there are marked differences among the magnitudes of these responses. These differences may be attributed to unique aspects of the relative positioning of the probes on the skin and under the bladder, causing differential sensitivities to the application of flow pressure within the bladder. In general, the application of pressure produces a reduction in measured blood flow in the underlying tissue, and release of pressure likewise produces an increase in flow.

Another feature of the response of blood perfusion to the application of cryotherapy is the relative rate at which the local flow drops as the temperature is initially reduced. At the risk of oversimplification, the rates of perfusion drop can be grouped into two general classes: slow and rapid. Slow rates of decrease are seen in FIGS. 17B and 19B, whereas rapid rates are seen in FIGS. 18B, 20B, 21B, 22B, 25B and 25B. Interestingly, FIGS. 23B and 24B show both slow and rapid rates of drop in blood perfusion among the different probes on the same subject during the same trial. These individual probes are located at different sites where there may be differentials in the applied thermal histories and in the local control of vasomotor regulation.

A major question concerning the cryotherapy induction of ischemia is whether there is a dose response effect, where the dose is characterized in terms of both time and temperature of exposure on the skin surface. As with most elicited biological responses to thermal stress, the application time and temperature during cryotherapy are not independent in their causative potentials for producing kinetic physiological reactions.

The control data in FIG. 27 for perfusion with water at the present skin temperature of 31° C. show no indication of a thermally induced vasoconstriction. During the last approximate 90 minutes of this trial in which the thermal insulating effect of the bladder and thermal barrier caused skin temperature to rise continuously with a concomitant increase in blood flow to 50% above the baseline value. This effect is in accordance with data in the literature for the affect of heating skin on inducing vasodilation.

FIG. 28B presents the response of blood flow to active local heating via passage of warm water through the bladder that was started during the period of post cooling vasoconstriction. There was an immediate and continuous increase in the rate of skin blood flow throughout the time of active heating, resulting in blood perfusion values that at one measurement site were well in excess of baseline. When the active heating was stopped, the blood perfusions leveled off at their peak values and were maintained until active cooling was resumed, at which time the cycle of deep vasoconstriction was again initiated, continuing into the post cooling period as always. An interpretation of this data is that induction of a large positive heat flux into the skin offsets the action of the factor(s) that cause persistent vasoconstriction during and following active cryotherapy cooling, and that these factors are reestablished with subsequent cryotherapy application.

We studied cryotherapy protocols in which the initial period of applied cooling lasted over a range of times from 1 minute (FIGS. 21A and 22A) to 80 minutes (FIG. 22A) to determine the exposure time effect on the dose response of blood perfusion to cryotherapy. It is well understood from the principles of heat transfer that during the surface cooling of a semi-infinite medium such as the skin and underlying tissues, the cold front will penetrate inwardly as a wave. Thus, the surface temperature will reduce progressively over time, and the extent of surface cooling will be much less for short periods of exposure than for longer periods. This effect is illustrated in FIG. 30 which plots the minimum skin temperature reached during the initial cooling period as a function of the duration of exposure to a cooling bladder with continuously circulating ice water.

The data shows that even very brief periods of cryotherapy are sufficient to lower the skin to a temperature wherein significant vasoconstriction occurs. FIG. 21B shows that for only 1 minute of cooling there can be a 25% to 50% depression of the local blood flow that lasts for more than three hours, at which point in time this particular experiment was terminated. FIG. 22B shows sequential cooling periods of 1, 2, and 3 minutes for which there is an accumulative temperature effect that precipitates a compounded long term diminution in the cutaneous blood flow.

One result of this data is that a reduction in local tissue temperature produces a depression in blood perfusion that persists long after the tissue is rewarmed from the cooling episode. It is anticipated that continuous cryotherapy protocols will hold the state of ischemia as long as cooling is applied. But, this study demonstrates that tissue ischemia is elicited by even brief exposures lasting as short as one minute to the level of cooling associated with typical cryotherapy devices. The implications for causation of tissue necrosis and neuropathy are serious and, we believe, warrant further investigation and a plan for remediation.

Of aspect shown from the data presented herein is that virtually all cryotherapy devices that are available commercially have inherent operating conditions that produce an ischemic state having the potential for leading to injuries such as tissue necrosis and neuropathy. The results demonstrate that even short episodes of cryotherapy can produce a prolonged ischemia in the local area of treatment. The state of ischemia endures long after local temperatures have rewarmed toward the normal range, indicating that the existence of ischemia is not directly coupled to maintenance of a cold state. It is likely that a long acting humoral agent is released locally with a continuing vasoconstrictive effect long after tissue temperatures have rewarmed toward their baseline values. We anticipate that further research to confirm, identify and characterize the action of local vasomotive agents will aid in understanding and controlling the phenomenon of cryotherapy-induced tissue ischemia. It would appear that there is a significant opportunity to improve the practice of cryotherapy by reducing or eliminating the risk of the provoked persistent, long term ischemia without compromising the therapeutic efficacy of the technique.

Described herein are methods with associated devices to solve the problem caused by cryotherapy producing a state of persistent and profound ischemia that can lead to injury causation via tissue necrosis and neuropathy. To be successful, the process combines mitigation of the injury causation with retaining the therapeutically beneficial advantages of the cold treatment. Cryotherapy is thought to provide a favorable affect to injured tissues by pain analgesia and diminished swelling and inflammation. The latter may result from cold induced vasoconstriction that issues in reduced fluid extravasation in conjunction with a diminished rate and volume of blood flowing to tissue in the treatment area. Thus, it is thought to be important that for a net majority of the cryotherapy treatment time, it is desirable for the tissue temperature and blood perfusion rate both to be reduced below normal values. However, in order to avoid the progressive buildup of the conditions conducive to ischemic injury, it is necessary to create intermittent periods during which the tissues in the treatment area are perfused with a supply of fresh blood to provide the oxygen and nutrients requisite to support cell metabolism and to wash out accumulated metabolic by products in order to avoid cell death. In addition, a periodic perfusion with fresh blood will be conducive to the healing process. Given that cryotherapy is often prescribed for continuous use durations measured in consecutive days or longer, being able to maintain the foregoing balance is all the more critical to effective and safe cryotherapy. All embodiments set forth herein meet this set of criterion.

In order to function for its intended purpose, the disclosed devices overcome a deep state of vasoconstriction and persisting ischemia induced by cryotherapy on demand to return the blood flow to tissues to normal levels for a measured period of time that can be specified independently, and then to return the flow to the prior depressed levels for the sake of the cryotherapy efficacy. It will be shown from our human subject data that all of the present embodiments are fully functional according to this principle. There are five methods for inducing full blood perfusion from cold-induced vasoconstriction and then returning it to the vasoconstricted state are as follows.
1. Cyclic cooling and heating of the tissue in the treatment area.
2. Transcutaneous Electrical Nerve Stimulation (TENS) and Electrical Muscle Stimulation (EMS) of the tissue in the treatment area.
3. Kinematic motion of joints distal to or within the treatment area.
4. Impulse activated blood flow pumping.
5. Sequential Compression activated blood flow pumping.

The following data illustrate the action and effectiveness of each of these embodiments.

### 1. Cyclic Cooling and Heating of the Tissue in the Treatment Area.

This method consists of cooling the tissue for cryotherapy followed by active warming of the tissue for a defined period of time and then resumption of cooling to initiate another cycle. An example protocol is illustrated in FIG. 32. The controlled process is the temperature applied to the skin surface. The physiological response is characterized in terms of the blood perfusion in the treatment area. Note that during active cooling the perfusion drops rapidly. Likewise, during active warming, the perfusion rises rapidly. Conversely, during passive cooling and warming the perfusion remains in a largely static condition. Therefore, this method requires intervention with active cooling and warming to rapidly drive the local blood perfusion between high and low levels to achieve the cryotherapy effect and protection against ischemic injury. A feature of this method is that the active warming to overcome the cold-induced ischemic state does not block the subsequent return to deep vasoconstriction with the resumption of active cooling. Thus, the balance between cryotherapy effectiveness and injury prevention is achieved.

### 2. Transcutaneous Electrical Nerve Stimulation (TENS) and Electrical Muscle Stimulation (EMS) of the Tissue in the Treatment Area.

This method consists of applying oscillating electrical stimulation to the skin within the site of cooling to cause muscle contraction that stimulates blood flow. This phenomenon has been widely described in the literature as a method for causing an increase in blood flow in a target tissue. Readily commercially available devices can be applied for this purpose. Data from an example TENS protocol is shown in FIG. 33.

In this experiment, 2 different sets of TENS electrodes were applied at low frequency and high intensity to induce muscle contractions at 2 different sites proximal and distal to the knee. As it can be seen in the FIG. 33, there were a total of 3 TENS episodes each lasting for 5 minutes with a resting period of 15 minutes in between each 2 consecutive episodes. Each stimulation episode is associated with a spike increase in measured perfusion followed by return to the pre-stimulation values shortly after the end of stimulation. The difference in the extent of increase in perfusion detected by the 3 perfusion probes is to a great extent due to their relative localization. The TENS electrodes placed proximal to the knee were positioned medially on the lower portion of quadriceps, whereas the distal set was placed on the upper segment of anterior tibialis muscle. Thus, stimulation of the proximal set of electrodes created much stronger contractions and consequently the greater increase in perfusion signal. Perfusion probes, P2 and P3 were closer to the proximal and distal set of electrodes, respectively. Therefore, P2 detected a much higher increase in perfusion than P3 did. P1 was placed laterally on quadriceps and further away from the electrodes and, accordingly, recorded a smaller increase in perfusion.

At all perfusion measurement locations and for all TENS stimulations, the increase in perfusion was immediate with application of the stimulation, and the decrease to the prior ischemic state was also immediate upon withdrawal of stimulation. This behavior is important for effective function of the invention to both provide a periodic inflow of fresh blood bearing oxygen and nutrients to treated tissue and to maintain an overall reduced level of perfusion to limit inflammation and swelling.

3. Kinematic Motion Distal or Proximal to or within the Treatment Area.

This method of stimulating blood flow is based on causing motion in affected limbs that include the treatment area. Of particular benefit is the kinematic motion of joints that can be caused by the operation of a device or by exercise of the subject. They may occur during ambulation or while nonambulatory. The important feature of this method is that the motion causes a transient increase in the blood flow due to mechanical action on the circulatory system. Example data illustrating the efficacy of this method are presented in FIG. 34.

An experiment was conducted in which the subject was in a supine position with the foot held to a pivoting plate that oscillates about a small angle causing alternating dorsiflexion and plantarflexion of the ankle. All of the work to produce the movement was supplied by the device so that the subject was not required to use any muscles and could be a passive participant in the experiment. During the cooling process there is the typical depression of perfusion at all three measurement locations under the cooling pad. Two episodes of mechanical stimulation are shown in which a device engaged the foot to cause alternating plantarflexion and dorsiflexion of the ankle through an angle of about 10° while the subject remained completely passive so as to avoid introducing artifacts. The cycle period for the movement was approximately two seconds, and the duration was about two minutes, although this time is desired to optimize the period and frequency of stimulation to provide the best combination of cryotherapy benefit and safety with minimal risk of injury. As is readily apparent in FIG. 34, the mechanical stimulation causes an immediate strong upregulation of blood flow upon the start of movement that continues through the duration of stimulation. Upon cessation of the stimulation, there is an immediate return to the previous level of vasoconstriction induced by the cryotherapy. Both effects are necessary and desirable for proper function of the invention.

### 4. Impulse Activated Blood Flow Pumping.

Another embodiment to cause brief increases in blood flow through tissues undergoing cryotherapy treatment is via the concurrent application of an impulse blood pump. This is a standard technology that is widely applied to avoid the occurrence of deep vein thrombosis in bed-ridden patients. In this experiment the inventors tested a Covidien Kendall Novamedix A-V Impulse System Model 6060 and Covidien Foot Cuffs as an adjuvant to a DonJoy Iceman cryotherapy unit. The data is reported in FIG. 35.

The temperature time plots in FIG. 35A follow the standard response during and following active cooling with the cryotherapy unit. Blood flow data and the impulse pump pressure profile are shown in FIG. 35B. Air pressure in the pneumatic cuff is rapidly elevated to a peak value of about 2.8 psi (145 mm Hg) following which it decreases in a two step process over about 3.5 seconds. The device is programmed from the factory to cause pressure pulses on a 21 second cycle, although in principle the frequency could be changed to other values. The best frequency to prevent ischemic injury and to support cryotherapy effectiveness can be determined from further experiments. The blood flow responses to the pressure impulse is shown at three locations in dedicated plots: distally on the great toe (red), at the distal end of the cryotherapy pad closest to the foot cuff (green), and at the proximal end of the pad furthest from the cuff (black). The two blood flow profiles closest to the foot cuff show biphasic responses to the impulse associate with the outflow of deoxygenated blood during pressurization and the inflow of fresh oxygenated blood during depressurization. The flow profile furthest from the foot cuff shows a less distinct reaction to the pressurization because the effects of the impulse are distributed over a larger vascular network and volume of tissue the further the measurement is made from the impulse input.

The impulse blood pump used in conjunction and coordination with the cryotherapy unit provides clean, periodic impulses in blood flow in tissues with cold-induced vasoconstriction. The flow increases and decreases in direct response to the action of the impulse pump, providing a periodic supply of fresh blood into the region of ischemia and maintaining the vasoconstriction to limit inflammation and swelling.

### 5. Sequential Compression Activated Blood Flow Pumping.

Yet another embodiment of the present invention applies a Sequential Compression Device (SCD) in conjunction with a cryotherapy unit to periodically produce a fresh flow of blood through tissue in a state of cryotherapy-induced ischemia. The experiment shown was conducted with a Covidien Kendall Model 6325 SCD operating simultaneous with a DonJoy Iceman cryotherapy unit applied to the ankle. The SCD pressure sleeve was applied to the lower leg between the knee and ankle. The sleeve consists of three circumferential air bladders, each connected to a pressure source and controller. The bladders are inflated sequentially from distal to proximal to mechanical force blood to move back toward the heart. For use with the cryotherapy device, our experience shows that the most effective stimulation of blood flow in the treatment tissue occurs when the SCD is positioned proximal to the cooling pad. Increases in blood flow occur during refilling of the vasculature after the pressure in the SCD is released.

The data in FIG. 36B were obtained during the rewarming phase of the trial when the tissue perfusion level was at about 50% of the precooling baseline. It can be clearly observed that there is a direct transient increase in blood flow in synchrony with the pressure cycles of the SCD, demonstrating a cause and effect relationship between action of the device and a transient increase in blood flow. As with the other embodiments, the blood flow increases only during stimulation, meeting the criteria for effective augmentation of cryotherapy to prevent the occurrence of long term uninterrupted ischemia that can lead to tissue injury, while retaining the benefits of cryotherapy in controlling inflammation and swelling.

The results demonstrate the even short episodes of cryotherapy can produced a prolonged duration of ischemia in the local area of treatment as well as in more distal tissues. The state of ischemia endures long after local temperatures have rewarmed toward the normal range, indicating the ischemia is not directly coupled to a cold state. It is likely that a long acting humoral vasoconstrictive agent is released locally that continues to be active long after tissue temperatures have rewarmed toward their baseline values. Evidence for the action of such vasoconstrictive agents has been confirmed in the occurrence of nonfreezing cold injury. The suppression of blood perfusion at sites distal to the site of applied cryotherapy may be a consequence of the vasoconstrictive agent eventually washing downstream in the residual blood flow, causing distal vasoconstriction in tissues that were not affected directly by the cryotherapy.

In one embodiment, an apparatus that provides cooling and mechanical and/or electrical simulation to the treated site may be used to overcome the problems associated with cryotherapy. In one embodiment, a cryotherapy device for producing a cooling effect for treating tissue includes a temperature control device which alters the temperature of at least a portion of the tissue being treated during treatment; and a blood flow device that alters the blood flow rate through the portion of the tissue being treated during and following treatment.

In one embodiment the temperature control device includes a heat transfer substrate which receives a cooling fluid during treatment of the portion of the tissue and transfers heat between the cooling fluid and the tissue.

In one embodiment, the blood flow device provides mechanical stimulation to the tissue to increase the flow of blood through the portion of the tissue. In an embodiment, the mechanical stimulation is accomplished by an impulse to glabrous skin at the end of an appendage being treated. In an alternate embodiment, the blood flow device provides electrical stimulation to the tissue to increase or decrease the flow of blood through the portion of the tissue.

In an alternate embodiment, the temperature control device includes multiple segments that include a thermoelectric material. Each of the segments may be controlled individually, the segments being mounted on a flexible substrate that is capable of conforming to the surface geometry of the portion of the tissue. The multiple thermoelectric segments may be arranged and controlled so as to produce a temperature grid of cooler and warmer regions on the portion of the tissue that can be modulated in both position and time so as to produce a desired therapeutic cooling effect with reduced risk of causing induced injury to the treatment and adjacent areas.

The cryotherapy device may include a variety of sensors to monitor the conditions of the tissue being treated. For example, the cryotherapy device may include: one or more temperature sensors couplable to the tissue to measure the surface temperature of the tissue; one or more blood flow rate sensors couplable to the portion of the tissue to measure blood perfusion in the tissue; and one or more oxygenation sensors couplable to the portion of the tissue to measure the level of oxygenation in the tissue. The cryotherapy device may also include a controller coupled to one or more of the temperature sensors, one or more of the blood flow rate sensors and one or more of the oxygenation sensors. The controller may use the data collected from one or more of the sensors to modulate the operation of the temperature control device and the blood flow device to achieve a desired therapeutic outcome.

In another embodiment a device and method for applying cooling to the skin surface comprises a time/temperature history which effectively limits the extent of blood flow depression in the treatment area and distal areas.

In another embodiment, a device and method may apply mechanical massage to the treatment area to stimulate blood flow either continuously or periodically during cooling and in conjunction with the cooling effect.

In another embodiment, a device and method may combine active heating of the treatment area with active cooling. The combination of heating and cooling may be modulated in combinations of spatial and temporal patterns. The cooling and heating may be produced by a matrix of thermoelectric devices in thermal contact with the skin at a treatment area that can be modulated in patterns that vary in space and/or in time to produce a desired temperature effect and blood perfusion effect. The devices and methods may incorporate a thermal sensor in the area of therapy and a blood flow sensor in the area of therapy, both of which may be connected to a control function to regulate the cryotherapy system to achieve cooling benefit while avoiding unnecessary risk of causing ischemic injury.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments.

Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method for local therapeutic cooling of tissue comprising:
coupling a cryotherapy device to a portion of the tissue, wherein the cryotherapy device comprises a temperature control device and a blood flow device;
operating the temperature control device to alter the temperature of a portion of the tissue;
operating the blood flow device to alter the flow rate of blood through the portion of the tissue.

2. The method of claim 1, wherein the temperature control device comprises a heat transfer substrate, and wherein operating the temperature control device comprises:
supplying a cooling fluid to the heat transfer substrate; and
altering the flow rate of a cooling fluid being passed to the heat transfer substrate to control the temperature of the portion of the tissue.

3. The method of claim 1, wherein the temperature control device comprises a thermoelectric material, and wherein operating the temperature control device comprises applying voltage to the thermoelectric material to change the temperature of the thermoelectric material.

4. The method of claim 3, further comprising altering the applied voltage to thermoelectric material to create a pattern of heating and cooling of the portion of the tissue.

5. The method of claim 1, wherein the blood flow device provides mechanical stimulation to the tissue to increase the flow of blood through the portion of the tissue.

6. The method of claim 1, wherein the blood flow device provides electrical stimulation to the tissue to increase or decrease the flow of blood through the portion of the tissue.

7. The method of claim 1, further comprising applying heat to the portion of the tissue to alter the flow rate of blood through the portion of the tissue.

8. The method of claim 1, wherein the blood flow device is operated continuously for no longer than a predetermined period of time and/or for no more than a maximum percentage of a total treatment time and/or such that no longer than a predetermined period of time is allowed to pass between activation events.

9. The method of claim 1, wherein values for treatment temperature applied to the skin of the portion of the tissue and the time of application of the treatment temperature are predetermined.

10. The method of claim 9, wherein the treatment temperature applied to the skin is set to not be lower than a value that produces substantial ischemia in the tissues.

11. The method of claim 9, wherein the treatment temperature is selected such that the temperature of the tissue is in the range of 20° C.-22° C. or 24° C.-26° C.

12. The method of claim 9, further comprising heating the portion of the tissue to increase the blood flow rate through the portion of the tissue.

13. The method of claim 12, wherein the period of time during which the temperature of a region is raised is small in comparison to the time constant for heat diffusion into the tissue so as to limit the overall loss of therapeutic cooling benefit but is sufficiently long to produce a heating of the superficial layer of skin so as to elicit a temporary increase in blood perfusion through that region of skin.

14. The method of claim 12, wherein the blood flow device is operated to reduce the amount of edema and the occurrence of prolonged ischemia in the portion of the tissue being treated and/or in the surrounding tissue.

15. The method of claim 12, further comprising operating the temperature control device and the blood flow device independently.

16. The method of claim 12, further comprising operating the temperature control device to produce a temperature time history according to a predetermined pattern to achieve a targeted therapeutic outcome in which the risk of cold induced ischemia is reduced.

17. The method of claim 12, further comprising independently controlling the treatment temperature applied to the portion of the tissue and the time of application can be regulated independently.

18. A cryotherapy device for producing a cooling effect for treating tissue comprising:
a temperature control device which alters the temperature of at least a portion of the tissue being treated during treatment; and
a blood flow device that alters the blood flow rate through the portion of the tissue being treated during and following treatment.

19. The cryotherapy device of claim 18, wherein the temperature control device comprises a heat transfer substrate which receives a cooling fluid during treatment of the portion of the tissue and transfers heat between the cooling fluid and the tissue.

20. The cryotherapy device of claim 18, wherein the temperature control device comprises a thermoelectric material, and wherein the temperature of the thermoelectric material is altered by applying a voltage to the thermoelectric material.

21. The cryotherapy device of claim 20, further comprising a voltage source capable of supplying alternating applied voltage to thermoelectric material to create alternate heating and cooling of the portion of the tissue.

22. The cryotherapy device of claim 18, wherein the blood flow device provides mechanical stimulation to the tissue to increase the flow of blood through the portion of the tissue.

23. The cryotherapy device of claim 22, wherein the mechanical stimulation is accomplished by an impulse to glabrous skin at the end of an appendage being treated.

24. The cryotherapy device of claim 18, wherein the blood flow device provides electrical stimulation to the tissue to increase or decrease the flow of blood through the portion of the tissue.

25. The cryotherapy device of claim 18, further comprising a controller coupled to the temperature control device that regulates the temperature applied to the portion of the tissue being treated.

26. The cryotherapy device of claim 18, wherein the temperature control device comprises multiple segments comprising a thermoelectric material, wherein each of the segments can be controlled individually, the segments being mounted on a flexible substrate that is capable of conforming to the surface geometry of the portion of the tissue.

27. The cryotherapy device of claim 26, wherein the multiple thermoelectric segments are arranged and controlled so as to produce a temperature grid of cooler and warmer regions on the portion of the tissue that can be modulated in both position and time so as to produce a desired therapeutic cooling effect with reduced risk of causing induced injury to the treatment and adjacent areas.

28. The cryotherapy device of claim 18, further comprising one or more sensors couplable to a portion of the tissue, wherein the one or more sensors comprise:
temperature sensors that measure the surface temperature of the tissue; and/or
blood flow rate sensors that measure blood perfusion in the tissue, and/or
oxygenation sensors that measure the level of oxygenation in the tissue.

29. The cryotherapy device of claim 28, further comprising a controller coupled to one or more of the sensors.

30. The cryotherapy device of claim 29, wherein the controller records data obtained using one or more of the sensors.

31. The cryotherapy device of claim 30, wherein the data recorded by the controller is used by the controller to modulate the operation of the temperature control device and the blood flow device to achieve a desired therapeutic outcome.

32. A method for reducing the risk of cold-induced ischemic injury during cryotherapy by administering a composition comprising flavanols to a subject in need of cryotherapy, wherein the administered flavanols increase the bioavailability of nitric oxide, thereby attenuating the extent of vasoconstriction caused by local cooling of tissue.

33. A method for preventing ischemic injury during cyrotherapy comprising:
coupling a cryotherapy device to a portion of the tissue, wherein the cryotherapy device comprises a temperature control device and a blood flow stimulation device;
operating the temperature control device to alter the treatment temperature of a portion of the tissue; and
operating the blood flow stimulation device to increase the flow rate of blood through the portion of the tissue.

34. The method of claim 33, wherein the blood flow stimulation device is operated continuously for no longer than a predetermined period of time and/or for no more than a maximum percentage of a total treatment time and/or such that no longer than a predetermined period of time is allowed to pass between activation events.

35. The method of claim 33, wherein values for treatment temperature applied to the skin of the portion of the tissue and the time of application of the treatment temperature are predetermined.

36. The method of claim 35, wherein the treatment temperature applied to the skin is set to not be lower than a value that produces substantial ischemia in the tissues.

37. The method of claim 35, wherein the treatment temperature is selected such that the temperature of the tissue is in the range of 20° C.-22° C. or 24° C.-26° C.

38. The method of claim 35, wherein the treatment temperature is selected such that the temperature of the tissue is in the range of 12° C.-15° C. so as to control the level of pain in the treated tissues.

39. A method for preventing ischemic injury and thermal injury during cryotherapy comprising:
coupling a cryotherapy device to a portion of the tissue, wherein the cryotherapy device comprises a temperature control device, a heat transfer limiting thermal barrier, and a blood flow stimulation device;
operating the thermal barrier as sequential compression device periodically for which there are periods of time when the device is active interspersed with periods in which the device is inactive;
operating the temperature control device to alter the temperature of a portion of the tissue; and
operating the blood flow stimulation device to increase the flow rate of blood through the portion of the tissue.

40. The method of claim 39 wherein the heat transport properties of the thermal barrier ensure that the temperature of the skin surface will not fall below a predetermined minimum value.

41. A device for applying cryotherapy to a region of a mammal without causing a state of prolonged ischemia, comprising:
a cryotherapy device for applying to a portion of the tissue, wherein the cryotherapy device comprises a temperature control device, a thermal barrier device and a blood flow stimulation device;
a temperature control device that can modulate the temperature of the cryotherapy device to a designated value based on a feedback signal;
a thermal barrier device having designated heat conduction properties that can regulate the temperature drop between the cooling source in the cryotherapy device and the skin; and,
a blood flow stimulation device that can cause an increased flow of fresh blood to the tissue in the treatment region on demand and that can be operated periodically according to a designated pattern and that can be operated independently of the temperature control device feature so that blood flow may be stimulated during times when cooling is or is not actively applied to the treatment region so as to avoid the occurrence of prolonged periods of profound ischemia.

42. The device of claim 41 wherein the temperature control device causes cooling by a stream of refrigerated liquid.

43. The device of claim 41 wherein the feedback signal originates from one temperature sensor or more than one temperature sensor that is sensing temperatures alternatively or inclusively within a stream of refrigerated liquid associated with the cryotherapy device, on the surface of the cryotherapy device, and/or on the surface of the skin at the region of treatment.

44. The device of claim 41 wherein the thermal barrier is comprised of layers of a pneumatic bladder and of thermal insulating material which combine to provide a thermal resistance to the flow of heat from the skin to the cooling source of the thermal control device to limits the lowest temperature that the skin may reach. This thermal resistance may be designed for conditions of the thermal control device being set to it lowest operating temperature to ensure that the skin surface temperature does not drop below a determined value at steady state operation such as 25° C., 20° C., 15° C., or 10° C., or another determined temperature.

45. The device of claim 41 wherein the thermal barrier may have a sleeve like geometry or a linearly wrapped geometry that can be fitted circumferentially around a region of the body or a limb that is to be treated with cryotherapy.

46. The device of claim 41 wherein the thermal barrier may incorporate one or more pneumatic bladders that can be inflated and deflated sequentially in time in order to cause increased blood flow within the tissue of a treatment region and that can be repeated periodically.

47. The device of claim 41 wherein the blood flow stimulation device is applied to an area that is distal or proximal to the treatment area.

48. The device of claim 41 wherein the stimulation of blood flow is caused by the operation of the thermal barrier in a pattern comprising:
a period of causation of increased blood flow having a determined duration, such as 10 seconds, 30 seconds, 60 seconds, or another determined duration;
a frequency of causation of increased blood flow determined to occur at intervals of time that are longer than the duration of increased blood flow, such as 10 minutes, 20 minutes, 30 minutes, 60 minutes, or another determined frequency; and
a combination of the duration and frequency of causatively increased blood flow operates to have the net effects of reducing swelling in the treatment region and of prohibiting the occurrence of continuing ischemia for a period of time long enough such that the eventual reperfusion of the treatment region with fresh blood does not cause reperfusion injury.

49. A method for enhancing the healing of wounded tissue to which cryotherapy is applied comprising:
applying the cryotherapy device of claim 41 to the treatment region;
avoiding a condition of prolonged, persistent ischemia in the treatment region by application of the periodic blood flow stimulation feature of the device;
causing periodic perfusion of the tissues in the treatment region with blood having an elevated oxygen and nutrient content and therein washing accumulated metabolic byproducts from the tissue; and
continuing the periodic stimulation of blood flow through the treatment region after the cessation of active cooling by the cryotherapy device as may increase oxygen and nutrient concentrations and especially during periods of persistent ischemic.
